(19) United States
(12) Patent Application Publication
(10) Pub. No.: US 2001/0026939 A1
(43) Pub. Date: Oct. 4, 2001

(54) Insecticidal cotton plant cells

(76) Inventors: Douglas Rice, Des Moines, IA (US); Nadine Carozzi, Raleigh, NC (US); David M. Anderson, Placentia, CA (US); Kanniah Rajasekaran, Metairie, LA (US); Thirumale S. Rangan, Lubbock, TX (US); Richard L. Yenofsky, Arcadia, CA (US); Richard Lotstein, Carrboro, NC (US); Annick de Framond, Pittsboro, NC (US)

Correspondence Address: SYNGENTA, 3054 CORNWALLIS ROAD, RESEARCH TRIANGLE PARK, NC, 27709, US

(21) Appl. No.: 09/756,643

(22) Filed: Jan. 8, 2001

Related U.S. Application Data
(63) Continuation of application No. 08/218,697, filed on Mar. 28, 1994, now abandoned.
Application No. 08/218,697 is a continuation of application No. 07/759,969, filed on Sep. 16, 1991, now abandoned.
Application No. 07/759,969 is a continuation of application No. 07/274,452, filed on Nov. 18, 1988, now abandoned.
Application No. 07/274,452 is a continuation-in-part of application No. 07/122,109, filed on Nov. 18, 1987, now abandoned.

(51) Int. Cl. C12N 5/04

(52) U.S. Cl. 435/419

(57) ABSTRACT

Cotton cells are transformed with a chimeric gene that expresses in the cells a polypeptide having substantially the insect toxicity properties of Bacillus thuringiensis crystal protein. The transformed cells are regenerated into plants that are toxic to the larvae of lepidopteran insects.

This application is continuation-in-part of application Ser. No. 122,109, filed Nov. 18, 1987.

### BACKGROUND OF THE INVENTION

The present invention is directed to a chimeric gene that expresses in cotton cells insecticides having substantially the insect toxicity properties of the crystal protein produced by Bacillus thuringiensis.

Bacillus thuringiensis is a species of bacteria that produces a crystal protein, also referred to as delta-endotoxin. This crystal protein is, technically, a protoxin that is converted into a toxin upon being ingested by larvae of lepidopteran and dipteran insects.

The crystal protein from Bacillus thuringiensis is a potentially important insecticide having no known harmful effects on humans, other mammals, birds, fish or on insects other than the larvae of lepidopteran, coleopteran and dipteran insects. Other advantages of the use of the crystal protein from B. thuringiensis as an insecticide include its broad spectrum of activity against lepidopteran and dipteran insect larvae, and the apparent difficulty of such larvae to develop resistance against the crystal protein, even where the crystal protein is used on a large scale.

The crystal protein is effective as an insecticide when it is applied to plants subject to lepidopteran larvae infestation. Such plants include broccoli, lettuce and cotton. Lepidopteran larvae infestation is especially serious in cotton plants. Application of the crystal protein to plants has usually been accomplished by standard methods such as by dusting or spraying.

The use of the crystal protein as a commercial insecticide has, however, been inhibited by a number of disadvantages. For example, the protoxin remains on the surface of the plants being treated, where it is effective only against surface-feeding larvae, and where it is inactivated by prolonged exposure to ultraviolet radiation. This inactivation may be at least one cause of the general lack of persistance of the crystal protein in the environment. Accordingly, frequent and expensive application of the crystal protein is necessary.

By taking advantage of genetic engineering, a gene responsible for the production of a useful polypeptide can be transferred from a donor cell, in which the gene naturally occurs, to a host cell, in which the gene does not naturally occur; Cohen and Boyer, U.S. Pat. Nos. 4,237,224 and 4,468,464. There are, in fact, few inherent limits to such transfers. Genes can be transferred between viruses, bacteria, plants and animals. In some cases, the transferred gene is functional, or can be made to be functional, in the host cell. When the host cell is a plant cell, whole plants can sometimes be regenerated from the cell.

Genes typically contain regions of DNA sequences including a promoter and a transcribed region. The transcribed region normally contains a 5′ untranslated region, a coding sequence, and a 3′ untranslated region.

The promoter contains the DNA sequence necessary for the initiation of transcription, during which the transcribed region is converted into mRNA. In eukaryotic cells, the promoter is believed to include a region recognized by RNA polymerase and a region which positions the RNA polymerase on the DNA for the initiation of transcription. This latter region, which is referred to as the TATA box, usually occurs about 30 nucleotides upstream from the site of transciption initiation.

Following the promoter region is a sequence that is transcribed into mRNA but is not translated into polypeptide. This sequence constitutes the so-called 5′ untranslated region and is believed to contain sequences that are responsible for the initiation of translation, such as a ribosome binding site.

The coding region is the sequence that is just downstream from the 5′ untranslated region in the DNA or the corresponding RNA. It is the coding region that is translated into polypeptides in accordance with the genetic code. B. thuringiensis, for example, has a gene with a coding sequence that translates into the amino acid sequence of the insecticidal crystal protein.

The coding region is followed by a sequence that is transcribed into mRNA, but is not translated into polypeptide. This sequence is called the 3′ untranslated region and is believed to contain a signal that leads to the termination of transcription and, in eukaryotic mRNA, a signal that causes polyadenylation of the transcribed mRNA strand. Polyadenylation of the mRNA is believed to have processing and transportation functions.

Natural genes can be transferred in their entirety from a donor cell to a host cell. It is often preferable, however, to construct a gene containing the desired coding region with a promoter and, optionally, 5′ and 3′ untranslated regions that do not, in nature, exist in the same gene as the coding region. Such constructs are known as chimeric genes.

Genetic engineering methods have been described for improved ways of producing the crystal protein. For example, Schnepf et al, U.S. Pat. Nos. 4,448,885 and 4,467,036, describe plasmids for producing crystal protein in bacterial strains other then B. thuringiensis. These methods permit production of the crystal protein, but do not overcome the disadvantages of using the crystal protein as a commercial insecticide.

Suggestions have been made to clone B. thuringiensis toxin genes directly into plants in order to permit the plants to protect themselves; Klausner, A, Bio/Technology 2: 408-419 (1984). Adang et al, European Patent Application 142,924 (Agrigenetics), allege a method for cloning toxin genes from B. thuringiensis in tobacco and suggest protecting cotton the same way. Such a suggestion constitutes mere speculation, however, until methods for transforming cotton cells and regenerating plants from the cells are available. Such methods are described in U.S. patent application Ser. No. 122,200 filed Nov. 18, 1987 entitled “Regeneration and Transformation of Cotton”, assigned to Phytogen, and U.S. patent application Ser. No. 122,162 filed Nov. 18, 1987 entitled “Regenerating Cotton from Cultured Cells”, assigned to CIBA-GEIGY. U.S. patent applications Ser. No. 122,200 and Ser. No. 122,162 were filed the same day as the present application. The disclosure of methods for transforming cotton cells in Phytogen patent application Ser. No. 122,200 and for regenerating cotton plants in Phytogen and CIBA-GEIGY patent applications Ser. No. 122,200 and Ser. No. 122,162 are incorporated herein by reference.

A need exists for developing new methods for producing the crystal protein of B. thuringiensis in cells of cotton plants and for new methods of killing lepidopteran larvae by feeding them cells of cotton plants containing a B. thuringiensis crystal protein or a similar polypeptide.

### OBJECTS OF THE INVENTION

It is an object of the present invention to provide a method for producing in cotton cells a toxin that has substantially the insect toxicity properties of B. thuringiensis crystal protein.

It is a further object of the present invention to provide a method for killing lepidopteran larvae by feeding them cotton plant cells containing chimeric genes that express an insecticidal amount of a toxin having substantially the insect toxicity properties of B. thuringiensis crystal protein. The insecticidal cotton plant cells include those from whole plants and parts of plants as well as individual cotton cells in culture.

It is an additional object of the present invention to provide the genes and other DNA segments as well as the cells and plants associated with the above methods.

### SUMMARY OF THE INVENTION

These and other objects of the present invention have been achieved by providing chimeric genes capable of expressing in cotton cells a polypeptide having substantially the insect toxicity properties of Bacillus thuringiensis crystal protein (hereinafter, chimeric Bt toxin gene).

Additional embodiments of the present invention include the chimeric Bt toxin gene in vectors, bacteria, plant cells in culture, and plant cells in living plants, as well as methods for producing a toxin having substantially the insect toxicity properties of Bacillus thuringiensis crystal protein in cotton cells and methods for killing insects by feeding them cotton cells containing a gene that expresses such a toxin.

### DESCRIPTION OF THE DRAWINGS

FIG. 1. Construction of mp 19/Bt, a plasmid containing the 5′ end of the Bt protoxin gene.

FIG. 2. Construction of mp 19/Bt ca/del, a plasmid containing the CaMV gene VI promoter fused to the 5′ end of Bt protoxin coding sequence.

FIG. 3. Construction of p702/Bt, a plasmid having the 3′ coding region of the protoxin fused to the CaMV transcription termination signals.

FIG. 4. Construction of pBR322/Bt 14, containing the complete protoxin coding sequence flanked by CaMV promoter and terminator sequences.

FIG. 5. Construction of pRK252/Tn903/BglII.

FIG. 6. Construction of pCIB 5.

FIGS. 7 & 8. Construction of pCIB 4.

FIG. 9. Construction of p CIB 2.

FIG. 10. Construction of pCIB 10, a broad host range plasmid containing T-DNA borders and gene for plant selection.

FIG. 11. Construction of pCIB10/19SBt.

FIG. 12. Construction of PCIB 710.

FIG. 13. Construction of pCIB10/710.

FIG. 14. Construction of pCIB10/35SBt.

FIG. 15. Construction of pCIB10/35SBt(KpnI).

FIG. 16. Construction of pCIB10/35SBt(BclI).

FIG. 17. Construction of pCIB10/35SBt(607).

FIG. 18. Summary of procedure for the regeneration and transformation of cotton plants.

FIG. 19. Construction of pCIB1300, a plasmid having a chimeric gene containing the CaMV 35S promoter/AMV leader/Bt(Bcl)deletion/35S terminator.

FIGS. 20, 21, and 22: Construction of PCIB 1301, having a chimeric gene containing the cotton rbc-gx promoter/Bt(607deletion) coding sequence.

FIG. 23. Construction of pCIB1302, having a chimeric gene containing the cotton rbc-gY promoter/Bt(607deletion) coding sequence.

FIG. 24. Restriction map of the cotton genomic clones carrying rbc-gX and rbc-gY.

FIG. 25. Nucleotide and amino acid sequences of rbc-gY. The first ATG and methionine of the transit peptide are boxed in the figure.

FIG. 26. Nucleotide and amino acid sequences of rbc-gx. The first ATG and methionine of the transit peptide are boxed in the figure.

### DEPOSITS

Escherichia coli MC1061, pCIB10/35SBt . . . ATCC 67329

Escherichia coli HB101, pCIB/19SBt . . . ATCC 67330

Plasmid pLVlll . . . ATCC 40235

The first two deposits listed above were made on Feb. 27, 1987 and the third on May 14, 1986 in the American Type Culture Collection, Rockville, Md. in accordance with the Budapest Treaty.

Phage lambda/rbc-gY . . . ATCC 40486

Phage lambda/rbc-gX . . . ATCC 40487

These two deposits were made on Aug. 25, 1988 in the American Type Culture Collection.

### DETAILED DESCRIPTION

The present invention is directed to the production of a chimeric BT toxin gene. The cotton plant cells contemplated include cells from any and all cotton plants into which foreign DNA can be introduced, replicated and expressed. Some suitable examples of cotton plant species include Gossypium hirsutum, Gossypium arboreum, and Gossypium barbadense. Gossypium hirsutum is preferred, and may be of the stripper or picker types. Stripper and picker cotton differ in their method of harvest, the stripper cotton bols being very firmly attached to the plant so that they are not released during late-season storms. Harvesting stripper cotton virtually destroys the plant. Picker cotton is less firmly attached and is harvested by less disruptive means. Some commercially available varieties of G. hirsutum capable of being regenerated by the method of the present invention include Acala 1515-75, Coker 304, Coker 315, Coker 201, Coker 310, Coker 312, DP 41, DP 90, Lankart 57, Lankart 611, McNair 235, Paymaster 145, Stoneville 506, Stoneville 825, Tomcot SP 21-S, Acala SJ-2, Acala SJ-4, Acala SJ-5, Acala SJC-1, Acala GC 510, Acala SJC-22, Acala SJC-28, Acala SJC-30, Siokra, Acala B-1644, Acala B-1810, Acala B-2724, Funk 519-2, Funk FC 3008, Funk FC 3024, Funk C 1568R, Funk FC 2005, Funk C 0947B, Funk FC 2028, Funk FC 2017, Funk C 1379, DPL 50, DPL 20, DPL 120, DPL 775, Tx-CAB-CS and Paymaster HS 26.

The preferred varieties are Acala SJ-2, Acala SJC-1, Acala GC 510, Acala SJC-28, Acala SCJ-30, Acala B-1644 and Siokra.

Acala SJ-2, Acala GC 510, Acala B-1644, and Siokra are especially preferred.

The term “plant cell” refers to any cell derived from a cotton plant. Some examples of cells encompassed by the present invention include differentiated cells that are part of a living plant; undifferentiated cells in culture; the cells of undifferentiated tissue such as callus or tumors; seeds; embryos; propagules and pollen,

The chimeric gene of this invention contains a promoter region that functions efficiently in cotton plants and a coding region that codes for the crystal protein from B. thuringiensis or for a polypeptide having substantially the insecticidal properties of the crystal protein from B. thuringiensis. The coding sequence of the chimeric gene is not known to be associated with the promoter in natural genes.

The 5′ and/or 3′ untranslated regions may, independently, be associated in nature with either the promoter or the coding region, or with neither the promoter or the coding region. Preferably, either the 5′ or the 3′ untranslated region is associated with the promoter in natural genes, and most preferably both the 5′ and 3′ regions are associated with the promoter in natural genes.

One could not predict, based on the state of the art at the time this invention was made, that a chimeric gene could be stably and functionally introduced into cotton cells. It was even less predictable that such cells would express an insecticidal polypeptide at any level, and especially at sufficient levels to impart insecticidal properties to the cells.

In order to be considered insecticidal, the plant cells must contain an insecticidal amount of toxin having substantially the insecticidal activity of the crystal protein from Bacillus thuringiensis. An insecticidal amount is an amount which, when present in plant cells, kills insects or at least prevents a function necessary for growth, such as feeding. Accordingly, the plant cells of the present invention are able to withstand attacks by lepidopteran larvae without, or with less, application of crystal protein or other insecticides when compared with plant cells that do not contain a gene producing an insecticidal polypeptide.

### The Gene

The Transcription Control Sequences

The chimeric gene of this invention contains transcription control sequences comprising promoter and 5′ and 3′untranslated sequences that are functional in cotton plants. These sequences may, independently, be derived from any source, such as, for example, virus, plant or bacterial genes.

The virus promoters and 5′ and 3′ untranslated sequences suitable for use are functional in cotton plants and include, for example, plant viruses such as cauliflower mosaic virus. Cauliflower mosaic virus (CaMV) has been characterized and described by Hohn et al in Current Topics in Microbiology and Immunology, 96, 194-220 and appendices A to G (1982). This description is incorporated herein by reference.

CaMV is an unusual plant virus in that it contains double-stranded DNA. At least two CaMV promoters are functional in plants, namely the 19S promoter, which results in transcription of gene VI of CaMV, and the 35S promoter. The 19S promoter and the 35S promoter are the preferred plant virus promoters for use in the present invention.

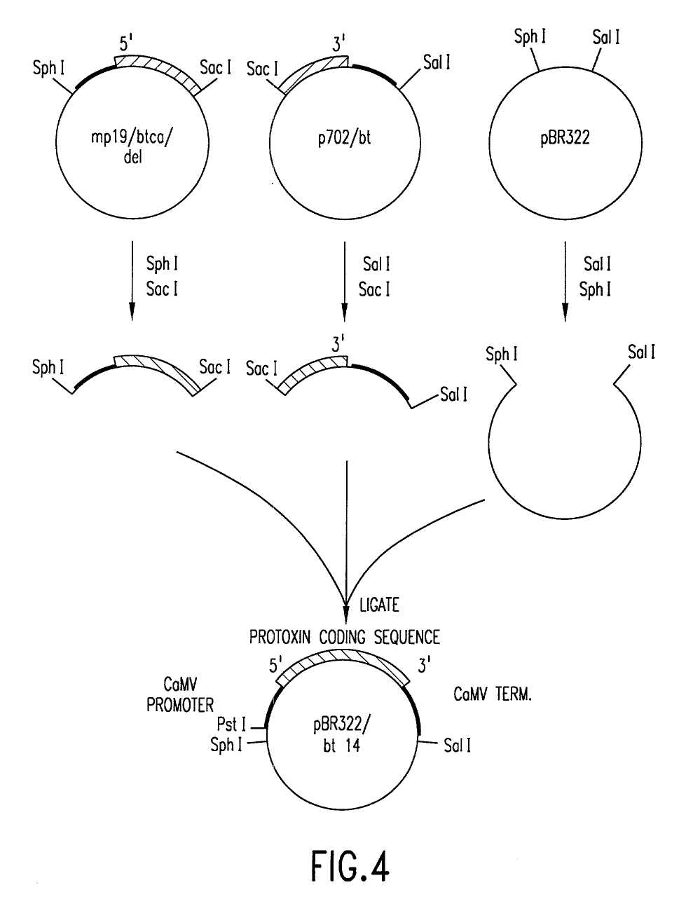

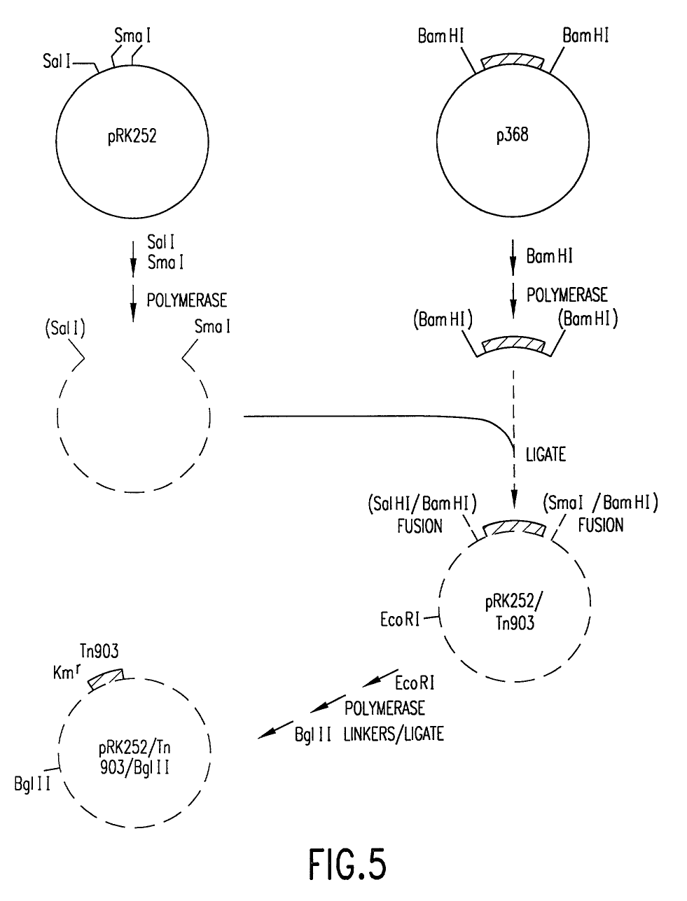

CaMV 19S promoters and 5′ untranslated regions may be obtained by means of a restriction map such as the map described in FIG. 4 on page 199 of the Hohn et al article mentioned above, or from the sequence that appears in Appendix C of the Hohn et al article.

In order to isolate the CaMV 19S promoter and, optionally, the adjacent 5′ untranslated region, a restriction fragment of the CaMV genome containing the desired sequences is selected. A suitable restriction fragment that contains the 19S promoter and the 5′ untranslated region is the fragment between the PstI site starting at position 5386 and the HindIII site starting at position 5850 of FIG. 4 and appendix C of the Hohn et al article.

By analgous methods, the 35S promoter from CaMV may be obtained, as is described in example 6 below.

Undesired nucleotides in the restriction fragment may optionally be removed by standard methods. Some suitable methods for deleting undesired nucleotides include the use of exonucleases (Maniatis et al, Molecular Cloning, Cold Spring Harbor Laboratory, Cold Spring Harbor, N.Y. pages 135-139 (1982)) and oligonucleotide-directed mutagenesis (Zoller et al, Methods in Enzymology, 100, 468 (1983)).

A similar procedure may be used to obtain a desirable 3′ untranslated region. For example, a suitable CaMV 19S gene 3′ untranslated sequence may be obtained by isolating the region between the EcoRV site at position 7342 and the BglII site at position 7643 of the CaMV genome as described in FIG. 4 and appendix C of the Hohn et al article.

Examples of plant gene promoters and 5′ and 3′ untranslated regions suitable for use in the present invention also include those of the gene coding for the small subunit of ribulose bisphosphate carboxylase and chlorophyl a/b-binding protein. These plant gene regions may be isolated from plant cells in ways comparable to those described above for isolating the corresponding regions from CaMV; see Morelli et al, Nature, 315, 200-204 (1985).

Suitable promoters and 5′ and 3′ untranslated regions from bacterial genes include those present in the T-DNA region of Agrobacterium plasmids. Some examples of suitable Agrobacterium plasmids include the Ti plasmid of A. tumefaciens and the Ri plasmid of A. rhizogenes. The Agrobacterium promoters and 5′ and 3′ untranslated regions useful in the present invention are, in particular, those present in the gene coding for octopine and nopaline synthase. These sequences may be obtained by methods similar to those described above for isolating CamV and plant promoters and untranslated sequences; see Bevan et al, Nature, 304, 184-187 (1983).

The Coding Region

The coding region of the chimeric gene contains a nucleotide sequence that codes for a polypeptide having substantially the toxicity properties of a Bacillus thuringiensis delta-endotoxin crystal protein. A polypeptide, for the purpose of the present invention, has substantially the toxicity properties of Bacillus thuringiensis delta-endotoxin crystal protein if it is insecticidal to a similar range of insect larvae as is the crystal protein from a subspecies of Bacillus thuringiensis. Some suitable subspecies include for example kurstaki, berliner, alesti, tolworthi, sotto, dendrolimus, tenebrionis, sandiego and aizawai. The preferred subspecies is kurstaki, and expecially kurstaki HDl.

The coding region may exist naturally in Bacillus thuringiensis. Alternatively, the coding region may contain a sequence that is different from the sequence that exists in Bacillus thuringiensis, but is equivalent because of the degeneracy of the genetic code.

The coding sequence of the chimeric gene may also code for a polypeptide that differs from a naturally occuring crystal protein delta-endotoxin but that still has substantially the insect toxicity properties of the crystal protein. Such a coding sequence will usually be a variant of a natural coding region. A “variant” of a natural DNA sequence is a modified form of the natural sequence that performs the same function. The variant may be a mutation, or may be a synthetic DNA sequence, and is substantially homologus to the corresponding natural sequence. A DNA sequence is substantially homologous to a second DNA sequence if at least 70%, preferably at least 80% and most preferably at least 90% of the active portions of the DNA sequence are homologous. Two different nucleotides are considered to be homologous in a DNA sequence of a coding region for the purpose of determining substantial homology if the substitution of one for the other constitutes a silent mutation.

The invention thus includes cotton cells and plants containing any chimeric gene coding for a sequence of amino acids having the insecticidal properties satisfying the requirements disclosed and claimed. It is preferred that the nucleotide sequence is substantially homologous at least to that portion or to those portions of the natural sequence responsible for insecticidal activity.

The polypeptide expressed by the chimeric gene of this invention will generally also share at least some immunological properties with a natural crystal protein, since it has at least some of the same antigenic determinants.

Accordingly, the polypeptide coded for by the chimeric gene of the present invention is preferably structurally related to the delta-endotoxin of the crystal protein produced by Bacillus thuringiensis. Bacillus thuringiensis produces a crystal protein with a subunit which is a protoxin having an Mr of about 130,000 to 140,000. This subunit can be cleaved by proteases or by alkali to form insecticidal fragments having an Mr as low as about 80,000, preferably about 70,000, more preferably about 60,000, and possibly even lower. The fragments preferably have a maximum Mr of about 120,000, more preferably about 110,000 and most preferably about 100,000. Chimeric genes that code for such fragments of the protoxin according to the present invention can be constructed as long as the fragments or portions of fragments have the requisite insecticidal activity. The protoxin, insecticidal fragments of the protoxin and insecticidal portions of these fragments can also be fused to other molecules such as polypeptides and proteins.

Coding regions suitable for use in the present invention may be obtained from crystal protein toxin genes isolated from Bacillus thuringiensis, for example, see Whitely et al., PCT application WO 86/01536 and U.S. Pat. Nos. 4,448,885 and 4,467,036. A preferred sequence of nucleotides that codes for a crystal protein is that shown in Formula I or a shorter sequence that codes for an insecticidal fragment of such a crystal protein. The disclosure of this sequence in Geiser et al., Gene 48: 109-118 (1986) is incorporated herein by reference.

The coding region of Formula I encodes the polypeptide of Formula II.

Formula I

| | |
|---|---|
| 1 | GTTAACACCC TGGGTCAAAA ATTGATATTT AGTAAAATTA GTTGCACTTT |
| 51 | GTGCATTTTT TCATAAGATG AGTCATATGT TTTAAATTGT AGTAATGAAA |
| 101 | AACAGTATTA TATCATAATG AATTGGTATC TTAATAAAAG AGATGGAGGT |
| 151 | AACTTATGGA TAACAATCCG AACATCAATG AATGCATTCC TTATAATTGT |
| 201 | TTAAGTAACC CTGAAGTAGA AGTATTAGGT GGAGAAAGAA TAGAAACTGG |
| 251 | TTACACCCCA ATCGATATTT CCTTGTCGCT AACGCAATTT CTTTTGAGTG |
| 301 | AATTTGTTCC CGGTGCTGGA TTTGTGTTAG GACTAGTTGA TATAATATGG |
| 351 | GGAATTTTTG GTCCCTCTCA ATGGGACGCA TTTCTTGTAC AAATTGAACA |
| 401 | GTTAATTAAC CAAAGAATAG AAGAATTCGC TAGGAACCAA GCCATTTCTA |
| 451 | GATTAGAAGG ACTAAGCAAT CTTTATCAAA TTTACGCAGA ATCTTTTAGA |
| 501 | GAGTGGGAAG CAGATCCTAC TAATCCAGCA TTAAGAGAAG AGATGCGTAT |
| 551 | TCAATTCAAT GACATGAACA GTGCCCTTAC AACCGCTATT CCTCTTTTTG |
| 601 | CAGTTCAAAA TTATCAAGTT CCTCTTTTAT CAGTATATGT TCAAGCTGCA |
| 651 | AATTTACATT TATCAGTTTT GAGAGATGTT TCAGTGTTTG GACAAAGGTG |
| 701 | GGGATTTGAT GCCGCGACTA TCAATAGTCG TTATAATGAT TTAACTAGGC |
| 751 | TTATTGGCAA CTATACAGAT CATGCTGTAC GCTGGTACAA TACGGGATTA |
| 801 | GAGCGTGTAT GGGGACCGGA TTCTAGAGAT TGGATAAGAT ATAATCAATT |
| 851 | TAGAAGAGAA TTAACACTAA CTGTATTAGA TATCGTTTCT CTATTTCCGA |
| 901 | ACTATGATAG TAGAACGTAT CCAATTCGAA CAGTTTCCCA ATTAACAAGA |
| 951 | GAAATTTATA CAAACCCAGT ATTAGAAAAT TTTGATGGTA GTTTTCGAGG |
| 1001 | CTCGGCTCAG GGCATAGAAG GAAGTATTAG GAGTCCACAT TTGATGGATA |
| 1051 | TACTTAACAG TATAACCATC TATACGGATG CTCATAGAGG AGAATATTAT |
| 1101 | TGGTCAGGGC ATCAAATAAT GGCTTCTCCT GTAGGGTTTT CGGGGCCAGA |
| 1151 | ATTCACTTTT CCGCTATATG GAACTATGGG AAATGCAGCT CCACAACAAC |
| 1201 | GTATTGTTGC TCAACTAGGT CAGGGCGTGT ATAGAACATT ATCGTCCACT |
| 1251 | TTATATAGAA GACCTTTTAA TATAGGGATA AATAATCAAC AACTATCTGT |
| 1301 | TCTTGACGGG ACAGAATTTG CTTATGGAAC CTCCTCAAAT TTGCCATCCG |
| 1351 | CTGTATACAG AAAAAGCGGA ACGGTAGATT CGCTGGATGA AATACCGCCA |
| 1401 | CAGAATAACA ACGTGCCACC TAGGCAAGGA TTTAGTCATC CATTAAGCCA |
| 1451 | TGTTTCAATG TTTCGTTCAG GCTTTAGTAA TAGTAGTGTA AGTATAATAA |
| 1501 | GAGCTCCTAT GTTCTCTTGG ATACATCGTA GTGCTGAATT TAATAATATA |
| 1551 | ATTCCTTCAT CACAAATTAC ACAAATACCT TTAACAAAAT CTACTAATCT |
| 1601 | TGGCTCTGGA ACTTCTGTCG TTAAAGGACC AGGATTTACA GGAGGAGATA |
| 1651 | TTCTTCGAAG AACTTCACCT GGCCAGATTT CAACCTTAAG AGTAAATATT |
| 1701 | ACTGCACCAT TATCACAAAG ATATCGGGTA AGAATTCGCT ACGCTTCTAC |
| 1751 | CACAAATTTA CAATTCCATA CATCAATTGA CGGAAGACCT ATTAATCAGG |
| 1801 | GGAATTTTTC AGCAACTATG AGTAGTGGGA GTAATTTACA GTCCGGAAGC |
| 1851 | TTTAGGACTG TAGGTTTTAC TACTCCGTTT AACTTTTCAA ATGGATCAAG |
| 1901 | TGTATTTACG TTAAGTGCTC ATGTCTTCAA TTCAGGCAAT GAAGTTTATA |
| 1951 | TAGATCGAAT TGAATTTGTT CCGGCAGAAG TAACCTTTGA GGCAGAATAT |
| 2001 | GATTTAGAAA GAGCACAAAA GGCGGTGAAT GAGCTGTTTA CTTCTTCCAA |
| 2051 | TCAAATCGGG TTAAAAACAG ATGTGACGGA TTATCATATT GATCAAGTAT |
| 2101 | CCAATTTAGT TGAGTGTTTA TCTGATGAAT TTTGTCTGGA TGAAAAAAAA |
| 2151 | GAATTGTCCG AGAAAGTCAA ACATGCGAAG CGACTTAGTG ATGAGCGGAA |
| 2201 | TTTACTTCAA GATCCAAACT TTAGAGGGAT CAATAGACAA CTAGACCGTG |
| 2251 | GCTGGAGAGG AAGTACGGAT ATTACCATCC AAGGAGGCGA TGACGTATTC |
| 2301 | AAAGAGAATT ACGTTACGCT ATTGGGTACC TTTGATGAGT GCTATCCAAC |
| 2351 | GTATTTATAT CAAAAAATAG ATGAGTCGAA ATTAAAAGCC TATACCCGTT |
| 2401 | ACCAATTAAG AGGGTATATC GAAGATAGTC AAGACTTAGA AATCTATTTA |
| 2451 | ATTCGCTACA ATGCCAAACA CGAAACAGTA AATGTGCCAG GTACGGGTTC |
| 2501 | CTTATGGCCG CTTTCAGCCC CAAGTCCAAT CGGAAAATGT GCCCATCATT |
| 2551 | CCCATCATTT CTCCTTGGAC ATTGATGTTG GATGTACAGA CTTAAATGAG |
| 2601 | GACTTAGGTG TATGGGTGAT ATTCAAGATT AAGACGCAAG ATGGCCATGC |
| 2651 | AAGACTAGGA AATCTAGAAT TTCTCGAAGA GAAACCATTA GTAGGAGAAG |
| 2701 | CACTAGCTCG TGTGAAAAGA GCGGAGAAAA AATGGAGAGA CAAACGTGAA |
| 2751 | AAATTGGAAT GGGAAACAAA TATTGTTTAT AAAGAGGCAA AAGAATCTGT |
| 2801 | AGATGCTTTA TTTGTAAACT CTCAATATGA TAGATTACAA GCGGATACCA |
| 2851 | ACATCGCGAT GATTCATGCG GCAGATAAAC GCGTTCATAG CATTCGAGAA |
| 2901 | GCTTATCTGC CTGAGCTGTC TGTGATTCCG GGTGTCAATG CGGCTATTTT |
| 2951 | TGAAGAATTA GAAGGGCGTA TTTTCACTGC ATTCTCCCTA TATGATGCGA |
| 3001 | GAAATGTCAT TAAAAATGGT GATTTTAATA ATGGCTTATC CTGCTGGAAC |
| 3051 | GTGAAAGGGC ATGTAGATGT AGAAGAACAA AACAACCACC GTTCGGTCCT |
| 3101 | TGTTGTTCCG GAATGGGAAG CAGAAGTGTC ACAAGAAGTT CGTGTCTGTC |
| 3151 | CGGGTCGTGG CTATATCCTT CGTGTCACAG CGTACAAGGA GGGATATGGA |
| 3201 | GAAGGTTGCG TAACCATTCA TGAGATCGAG AACAATACAG ACGAACTGAA |
| 3251 | GTTTAGCAAC TGTGTAGAAG AGGAAGTATA TCCAAACAAC ACGGTAACGT |
| 3301 | GTAATGATTA TACTGCGACT CAAGAAGAAT ATGAGGGTAC GTACACTTCT |
| 3351 | CGTAATCGAG GATATGACGG AGCCTATGAA AGCAATTCTT CTGTACCAGC |
| 3401 | TGATTATGCA TCAGCCTATG AAGAAAAAGC ATATACAGAT GGACGAAGAG |
| 3451 | ACAATCCTTG TGAATCTAAC AGAGGATATG GGGATTACAC ACCACTACCA |
| 3501 | GCTGGCTATG TGACAAAAGA ATTAGAGTAC TTCCCAGAAA CCGATAAGGT |
| 3551 | ATGGATTGAG ATCGGAGAAA CGGAAGGAAC ATTCATCGTG GACAGCGTGG |
| 3601 | AATTACTTCT TATGGAGGAA TAATATATGC TTTATAATGT AAGGTGTGCA |
| 3651 | AATAAAGAAT GATTACTGAC TTGTATTGAC AGATAAATAA GGAAATTTTT |
| 3701 | ATATGAATAA AAAACGGGCA TCACTCTTAA AAGAATGATG TCCGTTTTTT |
| 3751 | GTATGATTTA ACGAGTGATA TTTAAATGTT TTTTTTGCGA AGGCTTTACT |
| 3801 | TAACGGGGTA CCGCCACATG CCCATCAACT TAAGAATTTG CACTACCCCC |
| 3851 | AAGTGTCAAA AAACGTTATT CTTTCTAAAA AGCTAGCTAG AAAGGATGAC |
| 3901 | ATTTTTTATG AATCTTTCAA TTCAAGATGA ATTACAACTA TTTTCTGAAG |
| 3951 | AGCTGTATCG TCATTTAACC CCTTCTCTTT TGGAAGAACT CGCTAAAGAA |
| 4001 | TTAGGTTTTG TAAAAAGAAA ACGAAAGTTT TCAGGAAATG AATTAGCTAC |
| 4051 | CATATGTATC TGGGGCAGTC AACGTACAGC GAGTGATTCT CTCGTTCGAC |
| 4101 | TATGCAGTCA ATTACACGCC GCCACAGCAC TCTTATGAGT CCAGAAGGAC |
| 4151 | TCAATAAACG CTTTGATAAA AAAGCGGTTG AATTTTTGAA ATATATTTTT |
| 4201 | TCTGCATTAT GGAAAAGTAA ACTTTGTAAA ACATCAGCCA TTTCAAGTGC |
| 4251 | AGCACTCACG TATTTTCAAC GAATCCGTAT TTTAGATGCG ACGATTTTCC |
| 4301 | AAGTACCGAA ACATTTAGCA CATGTATATC CTGGGTCAGG TGGTTGTGCA |
| 4351 | CAAACTGCAG |

### Vectors

In order to introduce the chimeric gene of the present invention into plant cells, the gene is first inserted into a vector. If the gene is not available in an amount sufficient for transformation, the vector may be amplified by replication in a host cell. The most convenient host cells for amplification are bacterial or yeast cells. When a sufficient amount of the chimeric gene is available, it is introduced into cotton cells or tissue. The introduction of the gene into cotton plant cells or tissue may be by means of the same vector used for replication, or by means of a different vector.

Some examples of bacterial host cells suitable for replicating the chimeric gene include those of the genus Escherischia such as E. coli and Agrobacterium such as A. tumefaciens or A. rhizogenes. Methods for cloning heterologous genes in bacteria are described by Cohen et al, U.S. Pat. Nos. 4,448,885 and 4,467,036. The replication of genes coding for the crystal protein of Bacillus thuringiensis in E. coli is described in Wong et al., J. Biol. Chem. 258: 1960-1967 (1983).

The preferred bacterium host cell for amplifying the chimeric Bt genes of this invention is Agrobacterium. The advantage of amplifying the gene in Agrobacterium is that the Agrobacterium may then be used to insert the amplified gene into plant cells without further genetic manipulation.

Some examples of yeast host cells suitable for replicating the genes of this invention include those of the genus Saccharomyces.

Any vector into which the chimeric gene can be inserted and which replicates in a suitable host cell, such as in bacteria or yeast, may be used to amplify the genes of this invention. The vector may, for example, be derived from a phage or a plasmid. Some examples of vectors derived from phages useful in the invention include those derived from M13 and from lambda. Some suitable vectors derived from M13 include M13mp18 and M13mp19. Some suitable vectors derived from lambda include lambda-gt11, lambda-gt7 and lambda Charon 4.

Some vectors derived from plasmids expecially suitable for replication in bacteria include pBR322 (Bolivar et al, Gene, 2, 95-113 (1977); pUC18 and pUC19 (Norrander et al, Gene, 26, 101-106 (1983)); and Ti plasmids (Bevan et al., Nature, 304, 184-187 (1983)). The preferred vector for amplifying the gene in bacteria is pBR322.

Construction of Vectors for Replication

In order to construct a chimeric gene suitable for replication in bacteria, a promoter sequence, a 5′ untranslated sequence, a coding sequence and a 3′ untranslated sequence are inserted into or are assembled in the proper order in a suitable vector, such as a vector described above. In order to be suitable, the vector must be able to replicate in the host cell.

The promoter, 5′ untranslated region, coding region and 3′ untranslated region, which comprise the chimeric gene of the invention, may first be combined in one unit outside the vector, and then inserted into the vector. Alternatively, portions of the chimeric gene may be inserted into the vector separately. The vector preferably also contains a gene that confers a trait on the host cell permitting the selection of cells containing the vector. The preferred trait is antibiotic resistance. Some examples of useful antibiotics include ampicillin, tetracycline, hygromycin, G418, chloramphenicol, kanamycin, and neomycin.

Insertion or assembly of the gene in the vector is accomplished by standard methods such as the use of recombinant DNA [Maniatis, et al., Molecular Cloning, A Laboratory Manual, Cold Spring Harbor Laboratory, Cold Spring Harbor, N.Y. (1982)) and homologous recombination (Hinnen et al., Proc. Natl. Acad. Sci. USA, 75: 1929-1933 (1978)].

In recombinant DNA methods, the vector is cut, the desired DNA sequence is inserted between the cut pieces of the vector, and the ends of the desired DNA sequence are ligated to the corresponding ends of the vector.

The vector is most conveniently cut by means of suitable restriction endonucleases. Some suitable restriction endonucleases include those which form blunt ends, such as SmaI, HpaI and EcoRV, and those which form cohesive ends, such as EcoRI, SacI and BamHI.

The desired DNA sequence normally exists as part of a larger DNA molecule such as a chromosome, plasmid or transposon. The desired DNA sequence is excised from its source, and optionally modified so that the ends can be joined to the ends of the cut vector. If the ends of the desired DNA sequence and of the cut vector are blunt ends, they are joined by blunt end ligases such as T4 DNA ligase.

The ends of the desired DNA sequence may also be joined to the ends of the cut vector in the form of cohesive ends, in which case a cohesive-end ligase, which may also be T4 DNA ligase; is used. Other suitable cohesive-end ligases include, for example, E. coli DNA ligase.

Cohesive ends are most conveniently formed by cutting the desired DNA sequence and the vector with the same restriction endonuclease. In such a case, the desired DNA sequence and the cut vector have cohesive ends that are complementary to each other.

The cohesive ends may also be constructed by adding complementary homopolymer tails to the ends of the desired DNA sequence and to the cut vector using terminal deoxynucleotidyl transferase. Cohesive ends may also be constructed by adding a synthetic oligonucleotide sequence recognized by a particular restriction endonuclease, and cleaving the sequence with the endonuclease; see, for example, Maniatis et al, Molecular Cloning, a Laboratory Manual, Cold Spring Harbor Laboratory, Cold Spring Harbor, N.Y., 1982. Such synthetic oligonucleotide sequences are known as linkers.

Construction of Vectors for Transformation of Plants

The Bt toxin genes of the present invention may be introduced directly into plant cells by taking advantage of certain plasmids present in Agrobacterium. These plasmids contain regions that are naturally inserted into the genome of plant cells infected by Agrobacterium. The inserted region is called T-DNA (transferred-DNA). These plasmids, examples of which include the Ti (tumor inducing) plasmid of A. tumefacieus and the Ri (root inducing) plasmid of A. rhizogenes, contain T-DNA border sequences, at least one of which is believed to be necessary for the transfer of the T-DNA region from the plasmid to the genome of the infected plant cell. Natural Ti and Ri plasmids also contain virulence regions, the location of which is believed to be outside of the T-DNA region. The virulence regions are needed for the transfer of T-DNA to plant cells.

In modified systems the virulence regions may exist on plasmids different from the plasmid that contains the T-DNA. Such virulence region-containing plasmids are called helper plasmids.

The T-DNA regions that occur naturally are oncogenic and cause plant tumors. The oncogenic portions of these T-DNA regions may be partially or fully removed before, or simultaneously with, the insertion of the desired DNA sequence. The plasmids containing such modified T-DNA regions are said to be disarmed.

The genes suitable for use in the present invention are assembled in or are inserted into a T-DNA vector system by methods known in the art (Barton and Chilton, Methods in Enzymology, 101: 527 (1984) 1983; Chilton, “Plant Gene Vectors”, in “The Role of Plant Biotechnology in Plant Breeding”, Report of the 1984 Plant Breeding Research Forum, August 21-23 1984), Pioneer Hibred, page 177-192 (1985)). The T-DNA vector may be oncogenic (Hernalsteens et al, Nature, 287, 654 (1980)), partially disarmed (Barton et al, Cell, 32, 1033-1043 (1983)), fully disarmed (Zambryski et al, EMBO J., 2, 2143 (1983)), or may be based on artificial T-DNA vectors having synthetic T-DNA border-like sequences, (Wang et al, Cell, 38, 455 (1984)). Some suitable disarmed vectors containing T-DNA border regions include pGA 436, pGA 437 and pGA 438, as are described in An et al., EMBO J. 4: 277-284 (1985), pMON120; see Fraley et al, Proc. Natl. Acad. Sci. USA, 80, 4803-4807 (1983) and PCIB10; Rothstein et al., Gene 53, 153-161 (1987). The transfer of T-DNA is usually accomplished by incubating Agrobacterium with plant cell protoplasts or wounded plant tissue, see Caplan et al, Science, 222, 815 (1983).

In addition to the chimeric gene coding for a B. thuringiensis or a B. thuringiensis-like toxin, the vectors preferably further comprise a DNA sequence that permits the selection or screening of cotton plant cells containing the vector in the presence of cells that do not contain the vector. Such selectable or screenable markers may naturally be present in the vector into which the chimeric gene of this invention is introduced, or may be introduced into the vector either before or after the chimeric gene is introduced. Alternatively, the selectable or screenable marker gene or a portion thereof may first be joined to the desired chimeric gene or any portion thereof and the combined genes or gene segments may be introduced as a unit into the vector. The selectable or screenable marker may itself be chimeric.

The preferred selectable marker is a gene coding for antibiotic resistance. The gene must be capable of expression in the cells to be transformed. The cells can be cultured in a medium containing the antibiotic, and those cells containing the vector, which have an enhanced ability to survive in the medium, are selected. Genes that confer resistance to chloramphenicol, kanamycin, G418, hygromycin or, in principle, any other antibiotic may be useful as a selectable marker.

Some examples of genes that confer antibiotic resistance include, for example, those coding for neomycin phosphotransferase (kanamycin resistance, Velten et al, EMBO J., 3, 2723-2730 (1984)); hygromycin phosphotransferase [hygromycin resistance, van den Elzen et al., Plant Molecular Biology, 5, 299-302 (1985)] and chloramephenicol acetyltransferase.

An example of a gene useful primarily as a screenable marker in tissue culture for identification of plant cells containing genetically engineered vectors is a gene that encodes an enzyme having a chromogenic substrate. For example, if the gene encodes the enzyme beta-galactosidase, the plant cells are plated on a tissue culture medium containing the chromogenic substrate Xgal (5-chloro-4-bromo-3-indolyl-beta-D-galactoside), and under appropriate conditions, plant cells containing copies of this gene are strained blue by the dye indigo which is released when beta-galactosidase cleaves Xgal.

Introduction of Genes into Plants

The introduction of chimeric genes into plants in accordance with the present invention may be carried out with any T-DNA-derived vector system capable of introducing genes into cotton plant cells from Agrobacteria. The vector system may, for example, be a cointegrate system (Comai et al., Plasmid, 10, 21, (1983); Zambryski et al., EMBO J., 2, 2143 (1983) for example the split-end vector system (Fraley et al, Bio/Technology, 3, 629 (1985) as described by Chilton, “Plant Gene Vectors”, in “The Role of Plant Biotechnology in Plant Breeding”, Report of the 1984 Plant Breeding Research Forum, August 21-23, 1984, Pioneer Hibred, 177-192 (1985). The vector system may, on the other hand, be a binary system, de Framond et al, Bio/Technology, 1, 266 (1983); Hoekema et al, Nature, 303, 179 (1983), or a Ti plasmid engineered by homogenotization of the gene into the T-DNA, Matzke et al, J. Mol. Appl. Genet., 1, 39 (1981). A final possibility is a system wherein the T-DNA is on a plasmid and the virulence genes are on the chromosonal DNA.

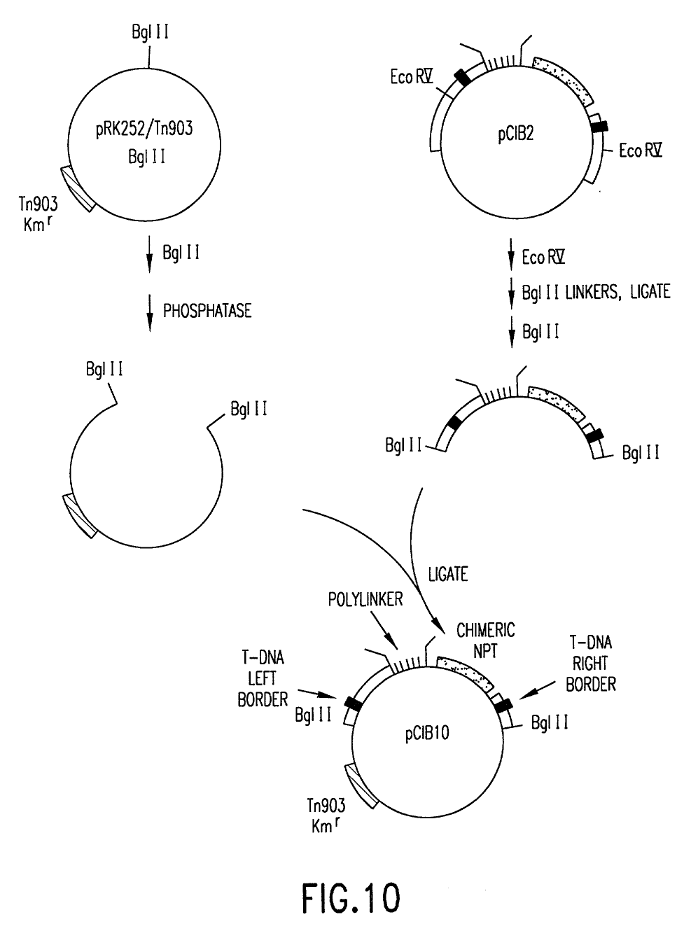

The preferred T-DNA vector system is a binary vector system, and especially a system utilizing pCIB10 Rothstein et al., Gene, 53, 153-161 (1987). (See FIG. 10).

The introduction of heterologous genes by recombinant DNA manipulation into a binary vector system is described by Klee et al, Bio/Technology, 3, 637 (1985). The insertion of genes into a T-DNA vector may be by homologous recombination using a double recombination strategy, Matzke et al, J. Mol. Appl. Genet., 1, 39 (1981); single recombination strategy, Comai et al, Plasmid, 10, 21 (1983); Zambryski et al, EMBO J., 2, 2143 (1983); or a single recombination strategy with no repeats in the T-DNA, Fraley et al, Bio/Technology, 3, 629 (1985) as described by Chilton, “Plant Gene Vectors”, in “The Role of Plant Biotechnology in Plant Breeding”, Report of the 1984 Plant Breeding Research Forum, Aug. 21-23, 1984, Pioneer Hibred, pages 177-192 (1985).

If the vectors containing the chimeric gene are not assembled in Agrobacterium, they may be introduced into Agrobacterium by methods known in the art. These methods include transformation and conjugation.

Transformation involves adding naked DNA to bacteria. Agrobacterium may be made susceptible to the introduction of naked DNA by freezing and thawing. The transformation of Agrobacterium is described by Holsters et al, Mol. Gen. Genet., 163, 181 (1978).

Conjugation involves the mating of a cell containing the desired vector, usually E. coli, with Agrobacterium. This method is described by Comai et al, Plasmid. 10, 21 (1983), and Chilton et al, Genetics, 83, 609 (1976).

The Agrobacterium spp. may be any strain of Agrobacterium capable of introducing genes into cotton plant cells. Some suitable examples include A. tumefaciens, A. rhizogeties, and A. radiobacter.

Genes are introduced into cotton plant cells by the method described in Phytogen's U.S. patent application Ser. No. 122,200, filed Nov. 18, 1987.

Transformed cotton plant cells containing the chimeric gene may be maintained in culture or may be regenerated into living plants. Expression is preferably of sufficient efficiency to render the plant cells insecticidal.

The medium capable of sustaining a particular plant cell in culture depends on the particular variety of cotton plant cell. For example, some suitable media include approximately 10 mg/l of 2,4 dichlorophenoxyacetic acid and either Murashige and Skoog inorganic salts (Physiol. Plant, 15: 473-497 (1962)] or Gamborg B-5 inorganic salts [Exp. Cell Res., 50: 151-158 (1968)].

The invention also includes living cotton plants, the cells of which contain the chimeric gene that expresses the polypeptide having substantially the insect toxicity properties of B. thuringiensis crystal protein.

Insecticides

The plant cells of this invention contain the chimeric gene and may be used to produce the polypeptide having substantially the insect toxicity of B. thuringiensis. The plant cells per se may constitute the insecticide. Plant cells used directly as insecticides may be cultured plant cells, or may be components of a living plant.

The toxin may also be isolated from the plant cells by known methods such as, for example, by extraction or chromatography. The extract, may be the total plant cell extract, a partially purified extract, or a pure preparation of the polypeptide. Any such extract or chromatographic isolate may be used in the same way as crystal protein from B. thuringiensis; see, for example, Deacon, Aspects of Microbiology, 7, Cole et al, ed, American Society for Microbiology (1983) and Miller et al, Science, 219, 715 (1983).

The present invention also includes a method for killing Lepidopteran larvae comprising feeding the larvae an insecticidal amount of cotton plant cells that contain the chimeric gene of the invention. The plant cells may be cultured plant cells, or may be components of living plants.

The present invention further includes cotton seeds of plants genetically engineered in accordance with this invention as long as the seeds contain the inserted gene and the desirable trait resulting therefrom. Progeny of plants produced by the method of this invention, including sexual and vegatative progeny, are futher embodiments. Sexual progeny may result from selfing or cross pollination.

### EXAMPLES

### Example 1

### General Recombinant DNA Techniques

Since many of the recombinant DNA techniques used in this invention are routine for those skilled in the art, a brief description of these commonly used techniques is included here rather than at each instance where they appear below. Except where noted, all of these routine procedures are described in the reference by Maniatis et al., “Molecular Cloning, A Laboratory Manual” (1982).

A. Restriction endonuclease digestions. Typically, DNA is present in the reaction mixture at approximately 50-500 ug/ml in the buffer solution recommended by the manufacturer, New England Biolabs, Beverly, Mass., 2-5 units of restriction endonucleases are added for each ug of DNA, and the reaction mixture incubated at the temperature recommended by the manufacturer for one to three hours. The reaction is terminated by heating to 65° C. for ten minutes or by extraction with phenol, followed by precipitation of the DNA with ethanol. This technique is also described on pages 104-106 of the Maniatis et al. reference.

B. Treatment of DNA with polymerase to create flush ends. DNA fragments are added to a reaction mixture at 50-500 ug/ml in the buffer recommended by the manufacturer, New England Biolabs. The reaction mixture contains all four deoxynucleotide triphosphates at a concentration of 0.2mM. The reaction is incubated at 15° C. for 30 minutes, and then terminated by heating to 65° C. for ten minutes. For fragments produced by digestion with restriction endonucleases that create 5′-protruding ends, such as EcoRI and BamHI, the large fragment, or Klenow fragment, of DNA polymerase is used. For fragments produced by endonucleases that produce 3′-protruding ends, such as PstI and SacI, T4 DNA polymerase is used. Use of these two enzymes is described on pages 113-121 of the Maniatis et al. reference.

C. Agarose gel electrophoresis and purification of DNA fragments from gels. Agarose gel electrophoresis is performed in a horizontal apparatus as described on pages 150-163 of Maniatis et al. reference. The buffer used is the Tris-borate buffer described therein. DNA fragments are visualized by staining with 0.5 ug/ml ethidium bromide, which is either present in the gel and tank buffer during electrophoresis or added following electrophoresis. DNA is visualized by illumination with short-wavelength or long-wavelength ultraviolet light. When the fragments are to be isolated from the gel, the agarose used is the low gelling-temperature variety, obtained from Sigma Chemical, St. Louis, Mo. After electrophoresis, the desired fragment is excised, placed in a plastic tube, heated to 65° C. for approximately 15 minutes, then extracted with phenol three times and precipitated with ethanol twice. This procedure is slightly modified from that described in the Maniatis et al. reference at page 170.

D. Addition of synthetic linker fragments to DNA ends. When it is desired to add a new restriction endonuclease site to the end of a DNA molecule, that molecule is first treated with DNA polymerase to create flush ends, if necessary, as described in the section above. Approximately 0.1-1.0 ug of this fragment is added to approximately 100 ng of phosphorylated linker DNA, obtained from New England Biolabs, in a volume of 20-30 ul containing 2 ul of T4 DNA ligase, from New England Biolabs, and 1 mM ATP in the buffer recommended by the manufacturer. After incubation overnight at 15° C., the reaction is terminated by heating the 65° C. for ten minutes. The reaction mixture is then diluted to approximately 100 ul in a buffer suitable for the restriction endonuclease that cleaves at the synthetic linker sequence, and approximately 50-200 units of this endonuclease are added. The mixture is incubated at the appropriate temperature for 2-6 hours, then the fragment is subjected to agarose gel electrophoresis and the fragment purified as described above. The resulting fragment will now have ends with termini produced by digestion with the restriction endonuclease. These termini are usually cohesive, so that the resulting fragment is now easily ligated to other fragments having the same cohesive termini.

E. Removal of 5′-terminal phosphates from DNA fragments. During plasmid cloning steps, treatment of the vector plasmid with phosphatase reduces recirculatization of the vector (discussed on page 13 of Maniatis et al. reference). After digestion of the DNA with the appropriate restriction endonuclease, one unit of calf intestine alkaline phosphatase, obtained from Boehringer-Mannheim, Indianapolis, Ind., is added. The DNA is incubated at 37° C. for one hour, then extracted twice with phenol and precitated with ethanol.

F. Ligation of DNA fragments. When fragments having complementary cohesive termini are to be joined, approximately 100 ng of each fragment are incubated in a reaction mixture of 20-40 ul containing approximately 0.2 units of T4 DNA ligase from New England Biolabs in the buffer recommended by the manufacturer. The incubation is conducted 1-20 hours at 15° C. When DNA fragments having flush ends are to be joined, they are incubated as above, except the amount of T4 DNA ligase is increased to 2-4 units.

G. Transformation of DNA into E. coli. E. coli strain HB101 is used for most experiments. DNA is introduced into E. coli using the calcium chloride procedure described by Maniatis et al. on pages 250-251. Transformed bacteria are selectively able to grow on medium containing appropriate antibiotics. This selective ability allows the desired bacteria to be distinguished from host bacteria not receiving transforming DNA. Determining what antibiotic is appropriate is routine, given knowledge of the drug resistance genes present on incoming transforming DNA and the drug sensitivity of the host bacteria. For example, where the host bacteria is know to be sensitive to ampicillin and there is a functional drug resistance gene for ampicillin on the incoming transforming DNA, ampicillin is an appropriate antibiotic for selection of transformants.

H. Screening E. coli for plasmids. Following transformation, the resulting colonies of E. coli are screened for the presence of the desired plasmid by a quick plasmid isolation procedure. Two convenient procedures are described on pages 366-369 of Maniatis et al. reference.

I. Large scale isolation of plasmid DNA. Procedures for isolating large amounts of plasmids in E. coli are found on pages 88-94 of the Maniatis et al. reference.

J. Cloning into M13 phage vectors. In the following description, it is understood that the double-stranded replicative form of the phage M13 derivatives is used for routine procedures such as restriction endonuclease digestions, ligations, etc.

### Example 2

### Construction of Chimeric gene in plasmid pBR322.

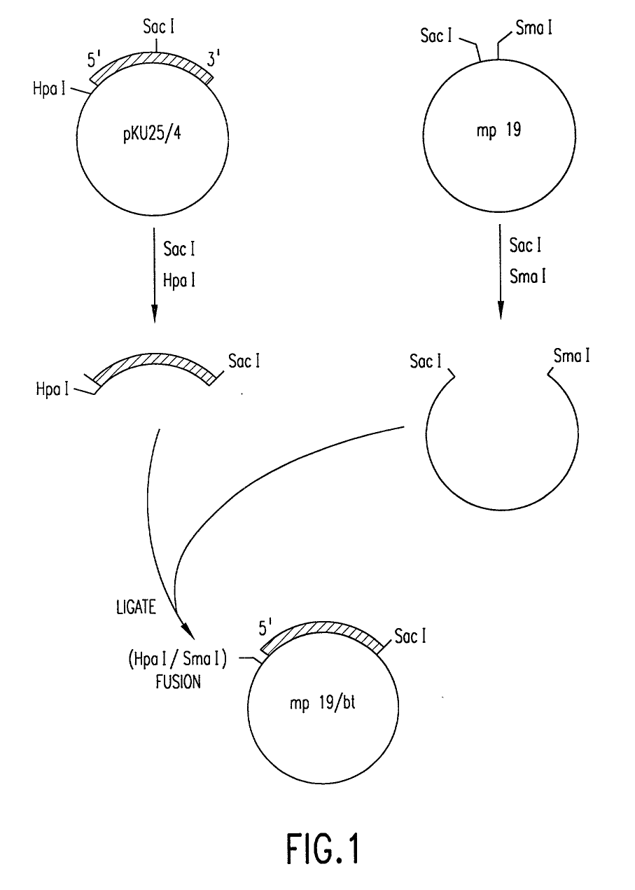

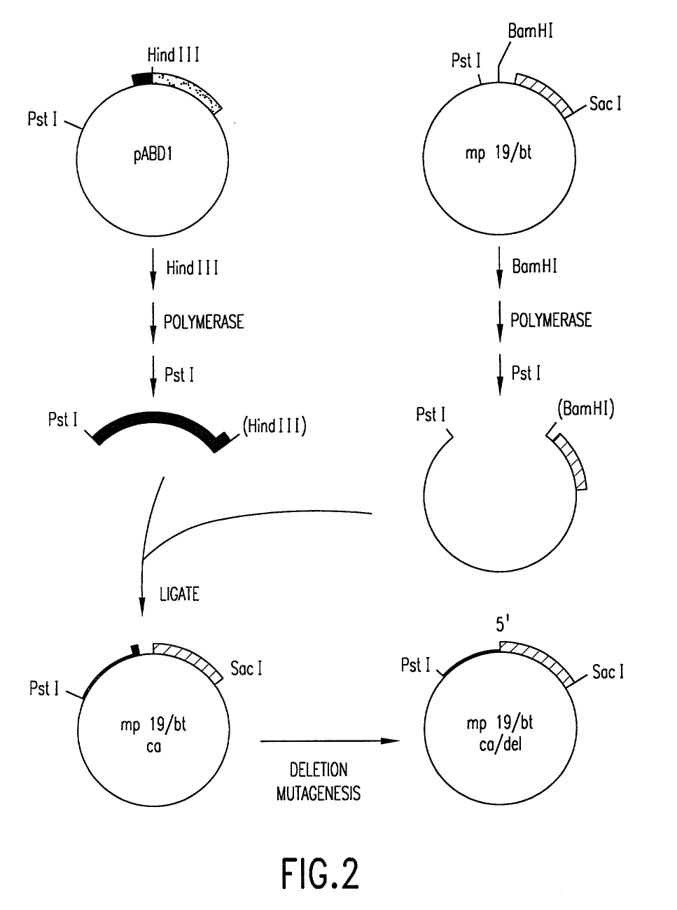

In order to fuse the CaMV gene VI promoter and protoxin coding sequences, a derivative of phage vector mp19 (Yanisch-perron et al., Gene 33: 103-119 (1985) is constructed. The following steps are shown in FIGS. 1 and 2. First, a DNA fragment containing approximately 155 nucleotides 5′ to the protoxin coding region and the adjacent approximately 1346 nucleotides of coding sequence are inserted into mp19. Phage mp19 ds rf (double-stranded replicative form) DNA is digested with restriction endonucleases SacI and SmaI and the approximately 7.2-kbp vector fragment is purified after electrophoresis through low-gelling temperature agarose by standard procedures. Plasmid pKU25/4, containing approximately 10 kbp (kilobase pairs) of Bacillus thuringiensis DNA, including the protoxin gene, is obtained from Dr. J. Nueesch, CIBA-GEIGY Ltd., Basel, Switzerland. The nucleotide sequence of the protoxin gene present in plasmid pKU25/4 is shown in formula I above. Plasmid pKU25/4 DNA is digested with endonucleases HpaI and SacI, and a 1503 bp fragment (containing nucleotides 2 to 1505 in Formula I) is purified as above. (This fragment contains approximately 155 bp of bacteria promoter sequences and approximately 1346 bp of the start of the protoxin coding sequence.) Approximately 100 ng of each fragment is then mixed, T4 DNA ligase added, and incubated at 15° C. overnight. The resulting mixture is transformed into E. coli strain HB101, mixed with indicator bacteria E. coli JM 101 and plated as described (messing, J., Meth. in Enzym. 101: 20-78 [1983]). One phage called mp19/bt is used for further construction below.

Next, a fragment of DNA containing the CaMV gene VI promoter, and some of the coding sequences for gene VI, is inserted into mp19/bt. Phage mp19/bt ds rf DNA is digested with BamHI, treated with the large fragment of DNA polymerase to create flush ends and recleaved with endonuclease PstI. The larger vector fragment is purified by electrophoresis as described above. Plasmid pABD1 is described in Paszkowski et al., EMBO J. 3, 2717-2722, (1984). Plasmid pABDl DNA is digested with PstI and HindIII. The fragment approximately 465 bp long containing the CaMV gene VI promoter and approximately 75 bp of gene VI coding sequence is purified. The two fragments are ligated and plated as described above. One of the resulting recombinant phages, called mp19/btca is used in the following experiment.

Phage mP19/btca contains CaMV gene VI promoter sequences, a portion of the gene VI coding sequence, approximately 155 bp of Bacillus thuringiensis DNA upstream of the protoxin coding sequence, and approximately 1346 bp of the protoxin coding sequence. To fuse the CaMV promoter sequences precisely to the protoxin coding sequences, the intervening DNA is deleted using oligonucleotide-directed mutagenesis of mp19/btca DNA. A DNA oligonucleotide with the sequence (5′) TTCGGATTGTTATCCATGGTTGGAGGTCTGA (3′) is synthesized by routine procedures using an Applied Biosystems DNA Synthesizer. This oligonucleotide is complimentary to those sequences in phage mp19/btca DNA at the 3′ end of the CaMV promoter (nucleotides 5762 to 5778 in Hohn, Current Topics in Microbiology and Immunology 96, 193-235 (1982) and the beginning of the protoxin coding sequence (nucleotides 156 to 172 in formula I above). The general procedure for the mutagenesis is that described in Zoller and Smith, Meth. in Enzym. 100: 468-500 (1983). Approximately five ug of single-stranded phage mp19/btca DNA is mixed with 0.3 ug of phosphorylated oligonucleotide in a volume of 40 ul. The mixture is heated to 65° C. for 5 min, cooled to 50° C., and slowly cooled to 4° C. Next, buffer, nucleotide triphosphates, ATP, T4 DNA ligase and large fragment of DNA polymerase are added and incubated overnight at 15° C. as described [see Zoller and Smith, Meth. in Enzym. 100: 468-500 (1983)]. After agarose gel electrophoresis, circular double-stranded DNA is purified and transfected into E. coli strain JM101. The resulting plaques are screened for sequences that hybridize with ³²P-labeled oligonucleotide, and phage are analyzed by DNA restriction endonuclease analysis. Among the resulting phage clones will be ones which have correctly deleted the unwanted sequences between the CaMV gene VI promoter and the protoxin coding sequence. This phage is called mp19/btca/del (see FIG. 2).

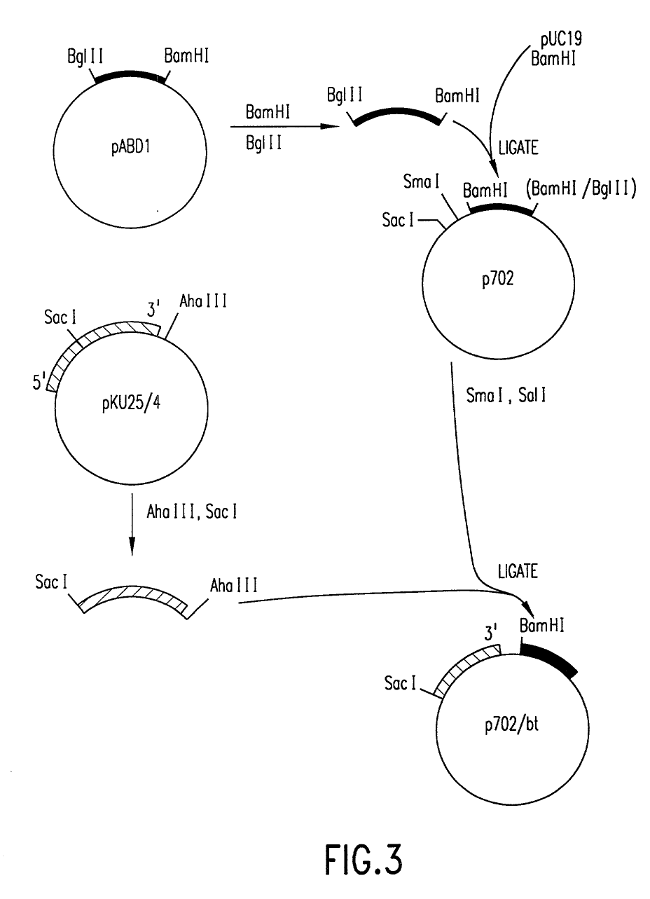

Next, a plasmid is constructed in which the 3′ coding region of the protoxin gene is fused to CaMV transcription termination signals. The following steps are shown in FIG. 3. First, plasmid pABDl DNA is digested with endonucleases BamHI and BglII and a 0.5 kbp fragment containing the CaMV transcription terminator sequences is isolated. Next plasmid pUC19, Yanisch-Perron et al., Gene 33: 103-119 (1985) is digested with BamHI, mixed with the 0.5 kbp fragment and incubated with T4 DNA ligase. After transformation of the DNA into E. coli strain HB101, one of the resulting clones, called plasmid p702, is obtained which has the structure shown in FIG. 3. Next, plasmid p702 DNA is cleaved with endonucleases SacI and SmaI, and the larger, approximately 3.2 kbp fragment is isolated by gel electrophoresis. Plasmid pKU25/4 DNA is digested with endonucleases AhaIII and SacI, and the 2.3-kbp fragment (nucleotides 1502 to 3773 in formula I above) containing the 3′ portion of the protoxin coding sequence (nt 1504 to 3773 of the sequence shown in Formula I) is isolated after gel electrophoresis. These two DNA fragments are mixed, incubated with T4 DNA ligase and transformed into E. coli strain HB101. The resulting plasmid is p702/bt (FIG. 3).

Finally, portions of phage mp19/btca/del ds rf DNA and plasmid p702/bt are joined to create a plasmid containing the complete protoxin coding sequence flanked by CaMV promoter and terminator sequences (see FIG. 4). Phage mp19/btca/del DNA is digested with endonucleases SacI and SphI, and a fragment of approx. 1.75 kbp is purified following agarose gel electrophoresis. Similarly, plasmid p702/bt DNA is digested with endonucleases SacI and SalI and a fragment of approximately 2.5 kbp is isolated. Finally, plasmid pBR322 DNA (Bolivar et al., Gene 2: 95-113 (1977) is digested with SalI and SphI and the larger 4.2-kbp fragment isolated. All three DNA fragments are mixed and incubated with T4 DNA ligase and transformed into E. coli strain HB101. The resulting plasmid, pBR322/bt14 is a derivative of pBR322 containing the CaMV gene VI promoter and translation start signals fused to the bacillus thuringiensis crystal protein coding sequence, followed by CaMV transcription termination signals (shown in FIG. 4).

3. Construction of a Ti plasmid-derived vector.

The vector pCIB10 (Rothstein et al., Gene, 53, 153-161 (1987)) is a Ti-plasmid-derived vector useful for transfer of the chimeric gene to plants via Agrobacterium tumefaciens. The vector is derived from the broad host range plasmid pRK252, which may be obtained from Dr. W. Barnes, Washington University, St. Louis, Mo. The vector also contains a gene for kanamycin resistance in Agrobacterium, from Tn903, and left and right T-DNA border sequences from the Ti plasmid pTiT37. Between the border sequences are the polylinker region from the plasmid pUC18 and a chimeric gene that confers kanamycin resistance in plants.

First, plasmid pRK252 is modified to replace the gene conferring tetracycline-resistance with one conferring resistance to kanamycin from the transposon Tn903 [Oka, et al., J. Mol. Biol., 147, 217-226 (1981)], and is also modified by replacing the unique EcoRI site in pRK252 with a BglII site (see FIG. 5 for a summary of these modifications). Plasmid pRK252 is first digested with endonucleases SalI and Smal, then treated with the large fragment of DNA polymerase I to create flush ends, and the large vector fragment purified by agarose gel electrophoresis. Next, plasmid p368, which contains Tn903 on an approximately 1050 bp BamHI fragment, is digested with endonuclease BamHI, treated with the large fragment of DNA polymerase, and an approximately 1050-bp fragment is isolated after agarose gel electrophoresis; this fragment contains the gene from transposon Tn903 which confers resistance to the antibiotic kanamycin [Oka et al., J. Mol. Biol., 147, 217-226 (1981)]. Plasmid p368 has been deposited with ATCC, accession number 67700. Both fragments are then treated with the large fragment of DNA polymerase to create flush ends. Both fragments are mixed and incubated with T4 DNA ligase overnight at 15° C. After transformation into E. coli strain HB101 and selection for kanamycin resistant colonies, plasmid pRK252/Tn903 is obtained (see FIG. 5).

Plasmid pRK252/Tn903 is digested at its unique EcoRI site, followed by treatment with the large fragment of E. coli DNA polymerase to create flush ends. This fragment is added to synthetic BglII restriction site linkers, and incubated overnight with T4 DNA ligase. The resulting DNA is digested with an excess of BglII restriction endonuclease and the larger vector fragment purified by agarose gel electrophoresis. The resulting fragment is again incubated with T4 DNA ligase to recircularize the fragment via its newly-added BglII cohesive ends. Following transformation into E. coli strain HB101, plasmid pRK252/Tn903/BglII is obtained (see FIG. 5).

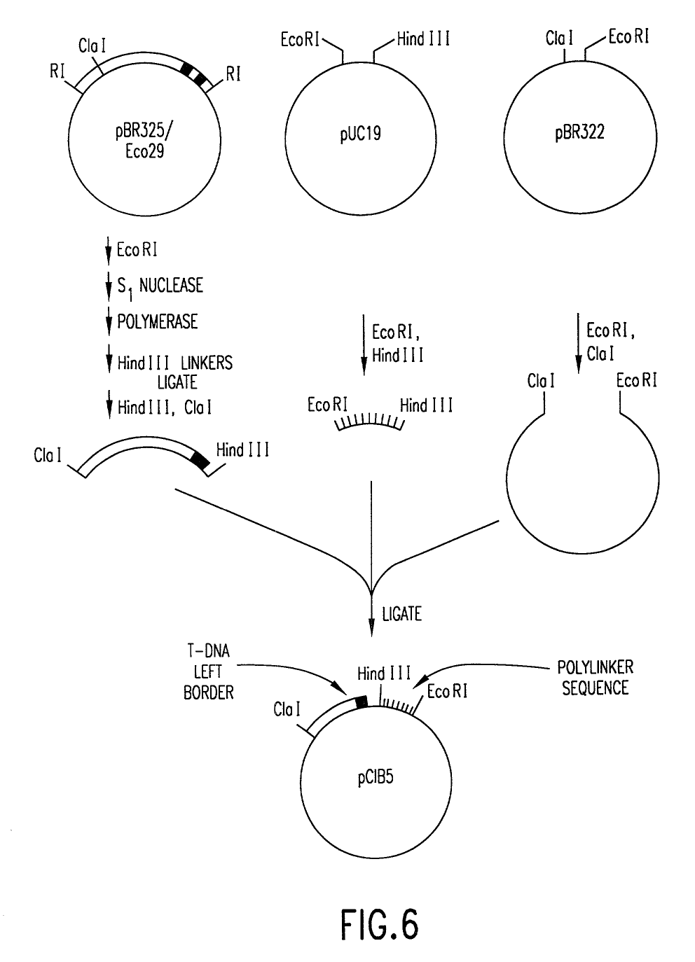

A derivative of plasmid pBR322 is constructed which contains the Ti plasmid T-DNA borders, the polylinker region of plasmid pUC19, and the selectable gene for kanamycin resistance in plants (see FIG. 6). Plasmid pBR325/Eco29 contains the 1.5-kbp EcoRI fragment from the nopaline Ti plasmid pTiT37. This fragment contains the T-DNA left border sequence; Yadav et al., Proc. Natl. Acad. Sci. USA, 79, 6322-6326 (1982). To replace the EcoRI ends of this fragment with HindIII ends, plasmid pBR325/Eco29 DNA is digested with EcoRI, then incubated with nuclease S1, followed by incubation with the large fragment of DNA polymerase to create flush ends, then mixed with synthetic HindIII linkers and incubated with T4 DNA ligase. The resulting DNA is digested with endonucleases ClaI and an excess of HindIII, and the resulting 1.1-kbp fragment containing the T-DNA left border is purified by gel electrophresis. Next, the polylinker region of plasmid pUC19 is isolated by digestion of the plasmid DNA with endonucleases EcoRI and HindIII and the smaller fragment (approx. 53 bp) is isolated by agarose gel electrophresis. Next, plasmid pBR322 is digested with endonucleases EcoRI and ClaI, mixed with the other two isolated fragments, incubated with T4 DNA ligase and transformed into E. coli strain HB101. The resulting plasmid, pCIB5, contains the polylinker and T-DNA left border in a derivative of plasmid pBR322 (see FIG. 6).

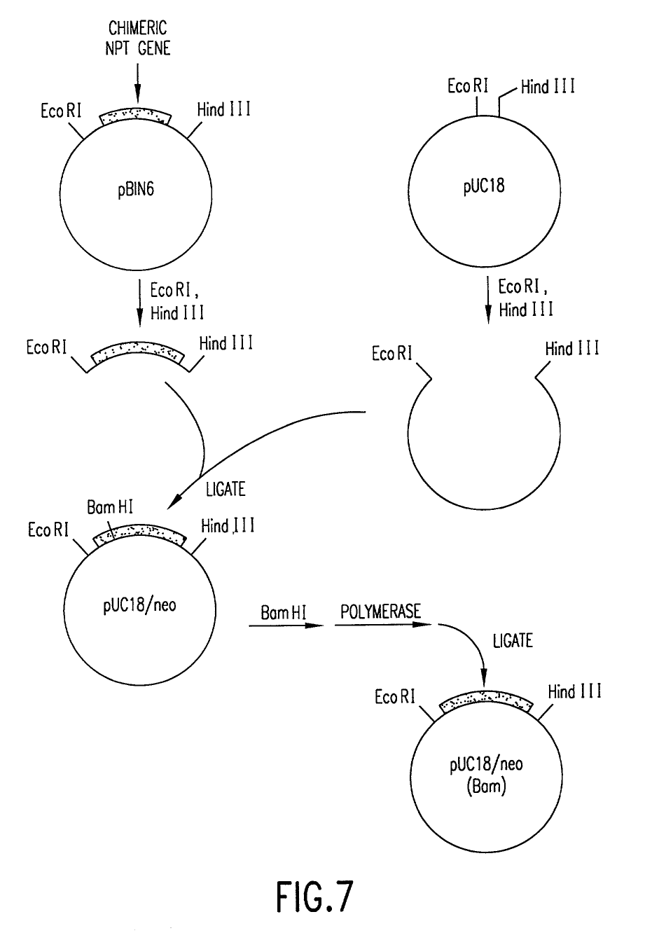

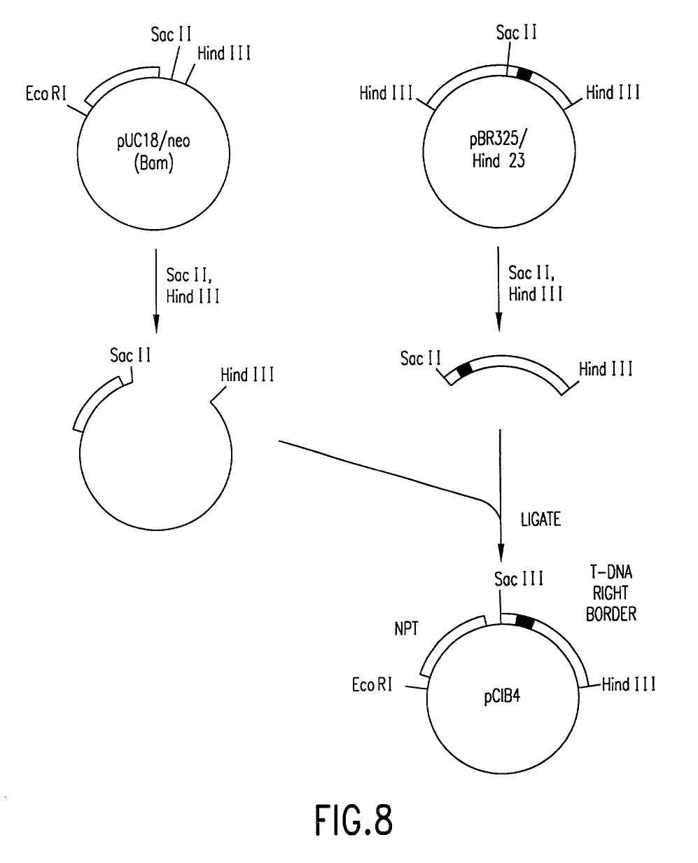

A plasmid containing the gene for expression of kanamycin resistance in plants is constructed (see FIGS. 7 and 8). Plasmid Bin6 is obtained from Dr. M. Bevan, Plant Breeding Institute, Cambridge, UK. This plasmid is described in the reference by Bevan, Nucl. Acids Res., 12, 8711-8721 (1984). Plasmid Bin6 DNA is digested with EcoRI and HindIII and the fragment approximately 1.5 kbp in size containing the chimeric neomycin phosphotransferase (NPT) gene is isolated and purified following agarose gel electrophoresis. This fragment is then mixed with plasmid pUC18 DNA which has been cleaved with endonucleases EcoRI and HindIII. Following incubation with T4 DNA ligase, the resulting DNA is transformed into E. coli strain HB101. The resulting plasmid is called pUC18/neo. This plasmid DNA contains an unwanted BamHI recognition sequence between the neomycin phosphotransferase gene and the terminator sequence for nopaline synthase; see Bevan, Nucl. Acids Res., 12, 8711-8721 (1984). To remove this recognition sequence, plasmid pUC18/neo is digested with endonuclease BamHI, followed by treatment with the large fragment of DNA polymerase to create flush ends. The fragment is then incubated with T4 DNA ligase to recircularize the fragment, and is transformed into E. coli strain HB101. The resulting plasmid, pUC18/neo(Bam) has lost the BamHI recognition sequence.

The T-DNA right border sequence is then added next to the chimeric NPT gene (see FIG. 8). Plasmid pBR325/Hind23 contains the 3.4-kbp HindIII fragment of plasmid pTiT37. This fragment contains the right T-DNA border sequence; Bevan et al., Nucl. Acids Res., 11, 369-385 (1983). Plasmid pBR325/Hind23 DNA is cleaved with endonucleases SacII and HindIII, and a 1.0 kbp fragment containing the right border is isolated and purified following agarose gel electrophoresis. Plasmid pUC18/neo(Bam) DNA is digested with endonucleases SacII and HindIII and the 4.0 kbp vector fragment is isolated by agarose gel electrophoresis. The two fragments are mixed, incubated with T4 DNA ligase and transformed into E. coli strain HB101. The resulting plasmid, pCIB4 (shown in FIG. 8), contains the T-DNA right border and the plant-selectable marker for kanamycin resistance in a derivative of plasmid pUC18.

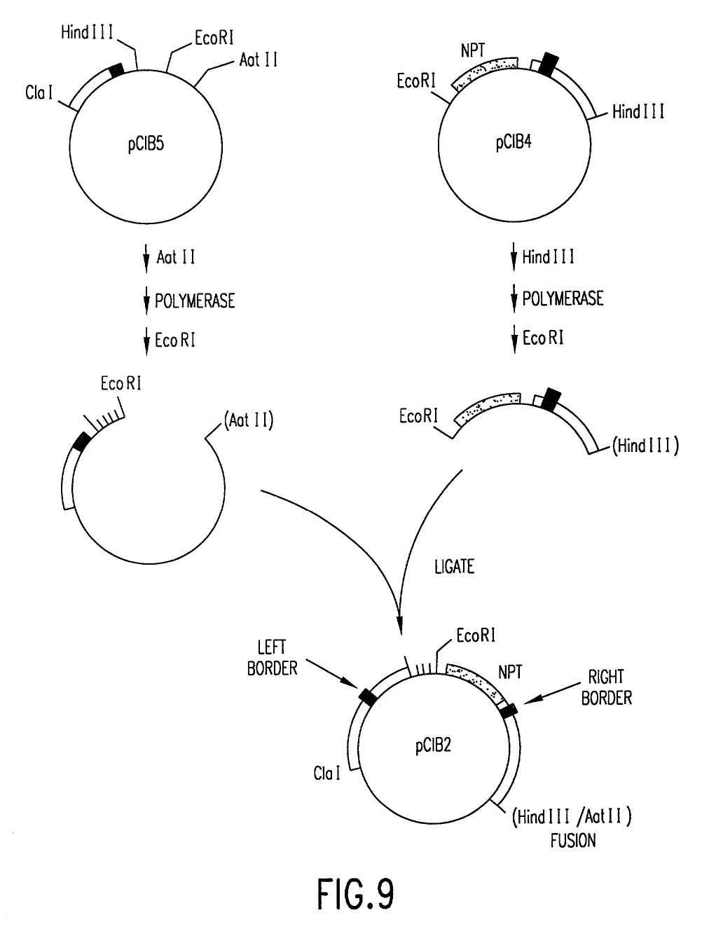

Next, a plasmid is constructed which contains both the T-DNA left and right borders, with the plant selectable kanamycin-resistance gene and the polylinker of pUC18 between the borders (shown in FIG. 9). Plasmid pCIB4 DNA is digested with endonuclease HindIII, followed by treatment with the large fragment of DNA polymerase to create flush ends, followed by digestion with endonuclease EcoRI. The 2.6-kbp fragment containing the chimeric kanamycin-resistance gene and the right border of T-DNA is isolated by agarose gel electrophoresis. Plasmid PCIB5 DNA is digested with endonuclease AatII, treated with T4 DNA polymerase to create flush ends, then cleaved with endonuclease EcoRI. The larger vector fragment is purified by agarose gel electrophoresis, mixed with the pCIB4 fragment, incubated with T4 DNA ligase, and transformed into E. coli Strain HB101. The resulting plasmid, pCIB2 (shown in FIG. 9) is a derivative of plasmid pBR322 containing the desired sequences between the two T-DNA borders.

The following steps complete construction of the vector pCIB10, and are shown in FIG. 10. Plasmid pCIB2 DNA is digested with endonuclease EcoRV, and synthetic linkers containing BglII recognition sites are added as described above. After digestion with an excess of BglII endonuclease, the approximately 2.6-kbp fragment is isolated after agarose gel electrophoresis. Plasmid pRK252/Tn903/BglII, described above (see FIG. 5), is digested with endonuclease BglII and then treated with phosphatase to prevent recircularization. These two DNA fragments are mixed, incubated with T4 DNA ligase and transformed into E. coli strain HB101. The resulting plasmid is the completed vector, pCIB10.

### Example 4

### Insertion of the chimeric protoxin gene into vector pCIB10.

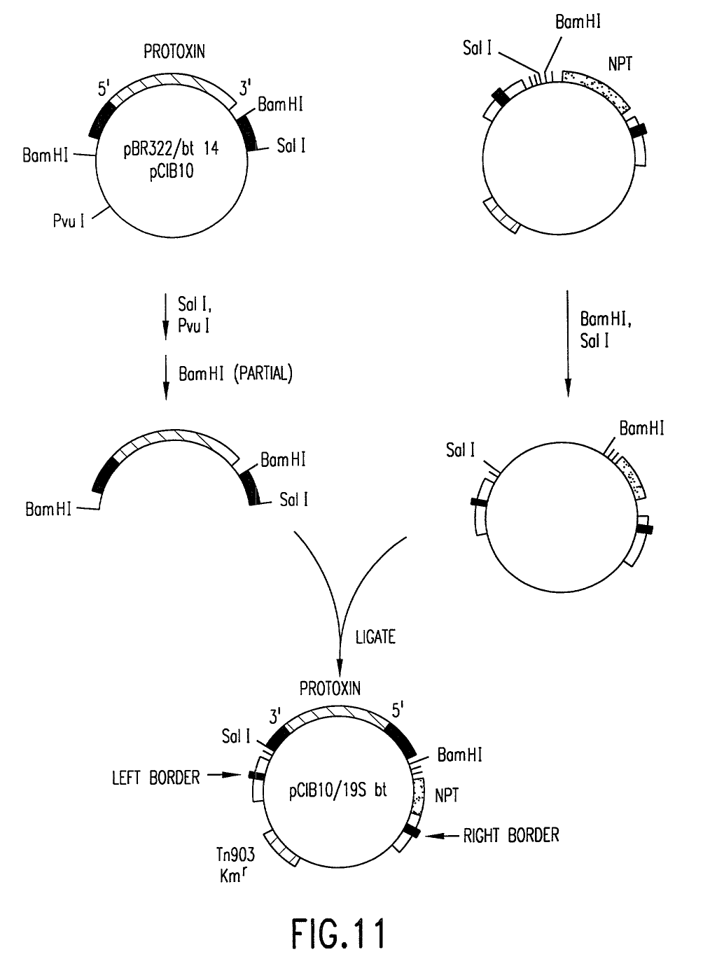

The following steps are shown in FIG. 11. Plasmid pBR322/Bt14 DNA is digested with endonucleases PvuI and SalI, and then partially digested with endonuclease BamHI. A BamHI-SalI fragment approximately 4.2 kbp in size, containing the chimeric gene, was isolated following agarose gel electrophoresis, and mixed with plasmid pCIB10 DNA which has been digested with endonucleases BamHI and SalI. After incubation with T4 DNA ligase and transformation into E. coli strain HB101, plasmid pCIB10/19SBt is obtained (see FIG. 11). This plasmid contains the chimeric protoxin gene in the plasmid vector pCIB10.

In order to transfer plasmid pCIB10/19SBt from E. coli HB101 to Agrobacterium, an intermediate E. coli host strain S17-1 is used. This strain, obtainable from Agrigenetics Research Corp., Boulder, Co., contains mobilization functions that can transfer plasmid pCIB10 directly to Agrobacterium via conjugation, thus avoiding the necessity to transform naked plasmid DNA directly into Agrobacterium (reference for strain S17-1 is Simon et al., “Molecular Genetics of the Bacteria-Plant Interaction”, A Puhler, ed, Springer Verlag, Berlin, pages 98-106, 1983). First, plasmid pCIB10/19SBt DNA is introduced into calcium chloride-treated S17-1 cells. Next, cultures of transformed S17-1 cells and Agrobacterium tumefaciens strain LBA4404 [Ooms et al., Gene, 14, 33-50 (1981)] are mixed and mated on an N agar (Difco) plate overnight at room temperature. A loopful of the resulting bacteria are streaked onto AB minimal media; Watson, B. et al., J. Bacteriol. 123: 255-264 (1975) plated with 50 ug/ml kanamycin and incubated at 28° C. Colonies are restreaked onto the same media, then restreaked onto N agar plates. Slow-growing colonies are picked, restreaked onto AB minimal media with kanamycin and single colonies isolated. This procedure selects for Agrobacteria containing the pCIBlO/19SBt plasmid.

### Example 5

### Transfer of the chimeric gene to tobacco plant cells.

Protoplasts of Nicotiana tabacum cv. “Coker 176” are prepared as follows. Four to five week old shoot cultures are grown aseptically in MS medium; Murashige and Skoog, Physiol. Plant., 15, 473-497 (1962) without hormones at 26° C. with a 16 hour light/8 hour dark photoperiod. Approximately 1.5 grams of leaf tissue are removed from the plant and distributed equally among 8-10 Petri dishes (100×25 mm, Lab-Tek), each containing 10 mls. of enzyme solution. Enzyme solution contains 1% cellulase R-10, obtained from Yakult Pharmaceutical Co., 0.25% macerase, from Calbiochem Co., 1% pectolyase Y-23, from Seishin Pharmaceutical Co., 0.45 M mannitol and 0.1X K3 salts; Nagy and Malign, Z. Pflanzenphysiol., 78, 453-455 (1976). Tobacco leaves are cut into thin strips with a scalpel, the dishes are sealed, placed on a gyrotory shaker at 35 rpm and incubated with the enzymes for 4-5 hours at room temperature.

Next, contents of the dishes are filtered through a cheesecloth-lined funnel and collected in a flask. The filtrate is pipetted into Babcock flasks containing 35 mls each of rinse solution. [Rinse solution contains 0.45M sucrose, MES (2-[N-morpholino]ethanesulfonic acid), and 0.1X K3 salts.) The bottles are centrifuged at 80 xg for ten minutes, after which the protoplasts will have floated to the top of the bottle. The protoplasts are removed with a 1 ml pipet, combined into one bottle, and rinsed twice more. The resulting protoplasts are suspended in K3 medium in a 15 ml disposable centrifuge tube. Concentration of protoplasts is determined by counting in a Fuchs-Rosenthal hemocytometer. Protoplasts are then plated at a density of 100,000/ml in 6 mls of liquid K3 medium per 100×20 mm Petri dish (Corning). The dishes containing the protoplasts are incubated at 26° C. in the dark for two days, during which time cell wall regeneration will occur.

After two-day incubation, 5 ul of a stationary culture of A. tumefaciens containing pCIB10/19SBt are added to the dish of protoplasts. (The agrobacteria are grown in YEP medium plus 50 ug/ml kanamycin at 28° C. until stationary phase is reached.) After incubation for three more days at 26° C., cefotaxime (Calbiochem) is added to 500 ug/ml to kill the Agrobacteria. The following day, cells are diluted with 3 mls fresh K3 medium per dish, and cefotaxime added again to a concentration of 500 ug/ml. Cells are then grown at 26° C. for 2-3 weeks and then screened on selective medium as described by DeBlock et al., EMBO J., 3, 1681-1689 (1984).

### Example 6

### Construction of a Bacillus thuringiensis protoxin chimeric gene with the CaMV 35S promoter.

Part I. Construction of CaMV 35S Promoter Cassette.

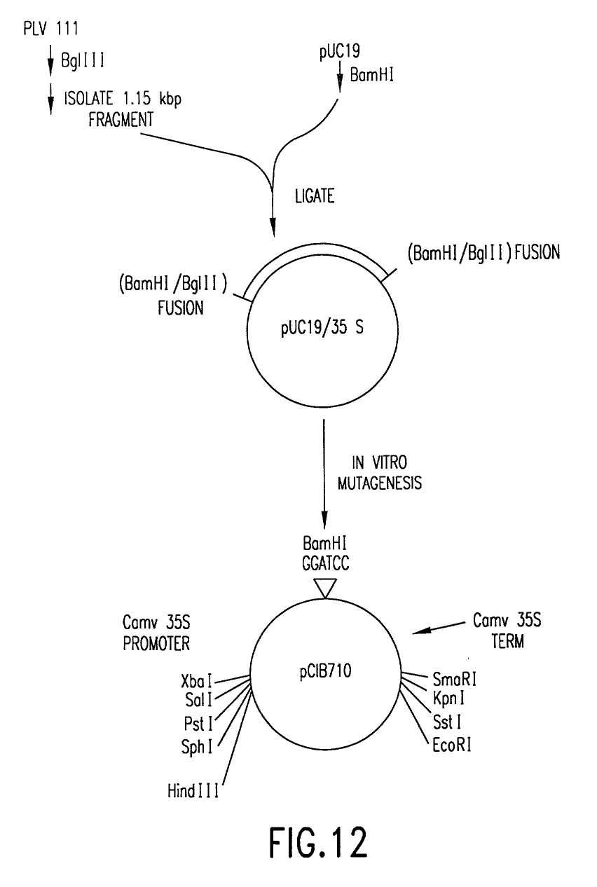

A CaMV 35S Promoter Cassette Plasmid pCIB710 is constructed as shown in FIG. 12. This plasmid contains CaMV promoter and transcription termination sequences for the 35S RNA transcript [Covey, S. N., Lomonossoff, G. P. and Hull, R., Nucl. Acids Res., 9, 6735-6747 (1981)]. A 1149 bp BglII restriction fragment of CaMV DNA [bp 6494-7643 in Hohn et al., Current Topics in Microbiology and Immunology, 96, 194-220 and Apendices A to G (1982)] is isolated from plasmid pLVlll (obtained from Dr. S. Howell, Univ. of California-San Diego; alternatively, the fragment can be isolated directly from CaMV DNA) by preparative agarose gel electrophoresis as described earlier and mixed with BamHI-cleaved plasmid pUC19 DNA, treated with T4 DNA ligase, and transformed into E. coli. (Note the BamHI restriction site in the resulting plasmid has been destroyed by ligation of the BglII cohesive ends to the BamHI cohesive ends.) The resulting plasmid, called pUC19/35S, is then used in oligonucleotide-directed in-vitro mutagenesis to insert the BamHI recognition sequence GGATCC immediately following CaMV nucleotide 7483 in the Hohn reference. The resulting plasmid, pCIB710, contains the CaMV 35S promoter region and transcription termination region separated by a BamHI restriction site. DNA sequences inserted into this BamHI site will be expressed in plants by these CaMV transcription regulation sequences. (Also note that pCIB710 does not contain any ATG translation initiation codons between the start of transcription and the BamHI site.)

Part II. Insertion of the CaMV 35S Promoter/Terminator Cassette into pCIB10.

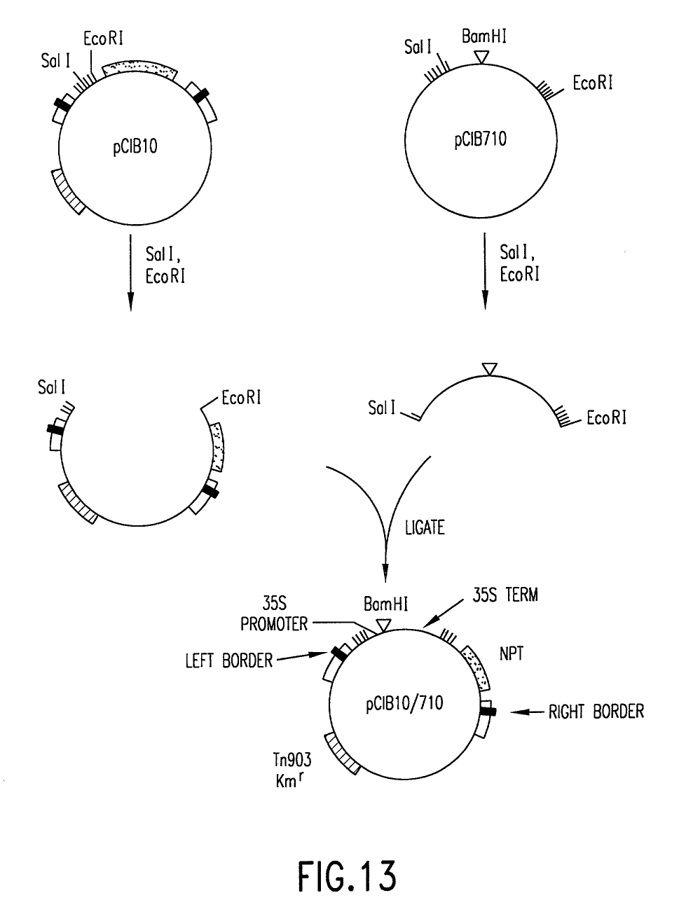

The following steps are outlined in FIG. 13. Plasmids pCIB10 and pCIB710 DNAs were digested with EcoRI and SalI, mixed and ligated. The resulting plasmid, pCIB10/710 has the CaMV 35S promoter/terminator cassette inserted into the plant transformation vector pCIB10. The CaMV-35S sequences are between the T-DNA borders in pCIB10, and thus will be inserted into the plant genome in plant transformation experiments.

Part III. Insertion of the Bacillus thuringiensis protoxin gene into pCIB10/710.

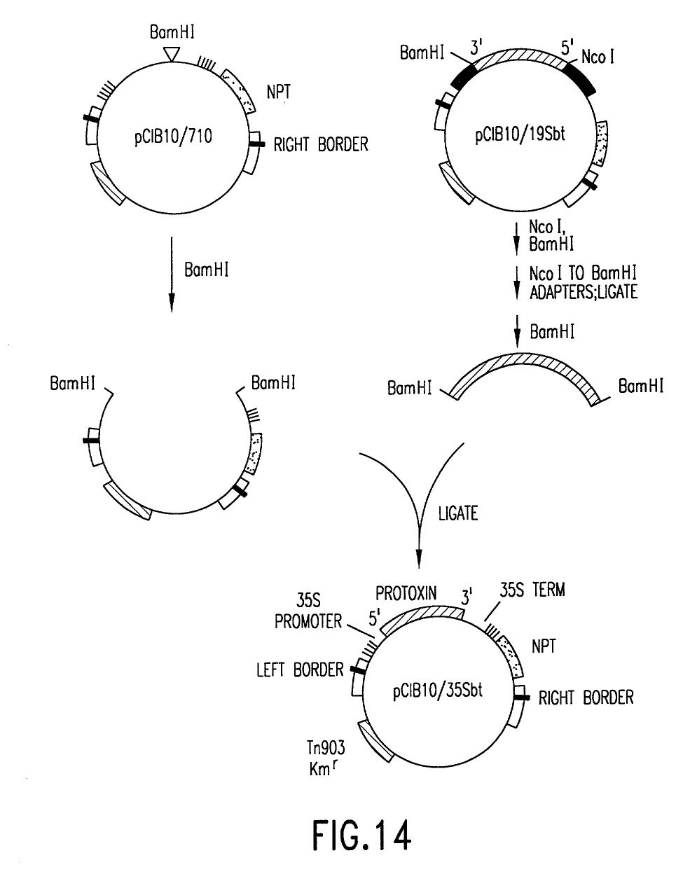

The following steps are outlined in FIG. 14. As a source of the protoxin gene, plasmid pCIB10/19SBt was digested with BamHI and NcoI, and the 3.6-kb fragment containing the protoxin gene was isolated by preparative gel electrophoresis. The fragment was then mixed with synthetic NcoI-BamHI adaptor with the sequence 5′-CATGGCCGGATCCGGC-3′, then digested with BamHI. This step creates BamHI cohesive ends at both ends of the protoxin fragment. This fragment was then inserted into BamHI-cleaved pCIB10/710. The resulting plasmid, pCIB10/35SBt, shown in FIG. 14, contains the protoxin gene between the CaMV 35S promoter and transcription termination sequences.

Part IV. Transfer of the plasmid pCIB10/35SBt into Agrobacterium tumefaciens for plant transformation.

The plasmid pCIB10/35SBt was transferred into A. tumefaciens strain LpA4404 as described in example 4, above.

### Example 6a

### Construction of pTOX, containing a chimeric gene encoding the insecticidal toxin gene of Bacillus thuringiensis var tenebrionis

A gene encoding the insecticidal crystal protein gene of Bacillus thuringiensis var. tenebrionis has been characterized and sequenced (Sekar, V. et al., Proc. Natl. Acad Sci USA, 84 (1987) 7036-7040]. This coding sequence is isolated on a convenient restriction fragment, such as a HindIII fragment of approximately 3 kb in size, and inserted into an appropriate plant expression vector, such as the plasmid PCIB 770 (Rothstein, S. et al., Gene, 53 (1987) 153-161]. The plasmid pCIB 770 contains a chimeric kanamycin gene for expression in plants, as well as the promoter and terminator of the 35S RNA transcript of CaMV (cauliflower mosaic virus) separated by a unique BamHl site. The restriction fragment bearing the toxin coding sequence is made compatible to the unique BamHI site of pCIB 770 by use of the appropriate molecular adapter and ligated together.

### Example 6b

### Construction of pSAN, containing a chimeric gene encoding the insecticidal toxin gene of Bacillus thuringiensis strain san diego

A gene encoding the insecticidal protein of Bacillus thuringiensis strain san diego has been characterized and sequenced by Herrnstadt et al., EP-0-202-739 and EP-0-213-818. This coding sequence is isolated on a convenient restriction fragment and inserted into the appropriate plant expression vector, such as PCIB 770. The plasmid pCIB770 contains a chimeric kanamycin gene for expression in plants, as well as the promoter and terminator of the 35S RNA transcript of CaMV [cauliflower mosaic virus] separated by a unique BamH site. The restriction fragment bearing the toxin coding sequence is made compatible to the unique BamHI site of pCIB 770 by use of the appropriate molecular adapter and ligated together.

### Example 7

### Construction of a deleted Bacillus thuringiensis protoxin gene containing approximately 725 amino acids, and construction of a chimeric gene containing this deleted gene with the CaMV 35S promoter

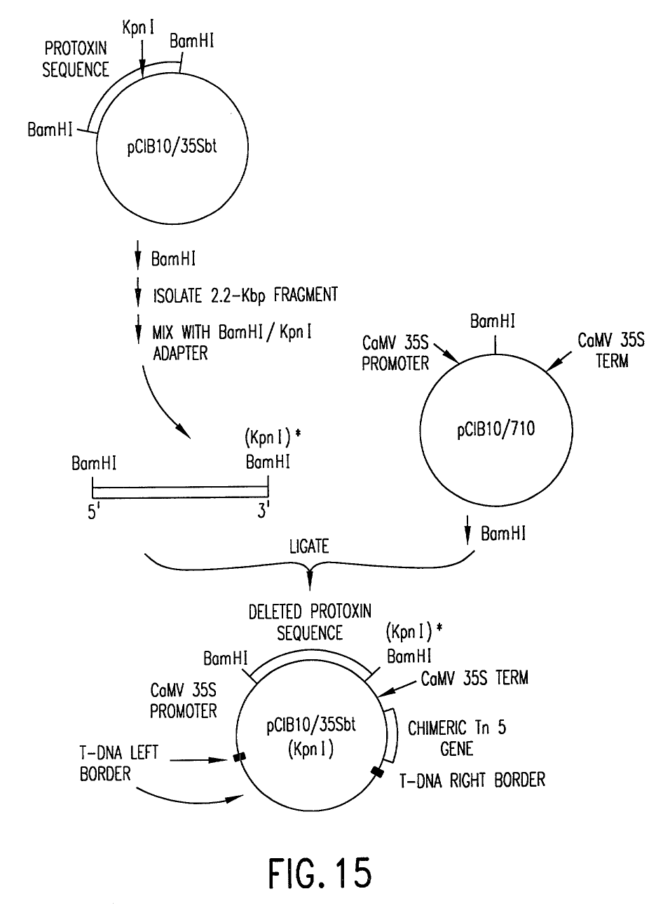

A deleted protoxin gene containing approximately 725 amino acids is made by removing the COOH-terminal portion of the gene by cleaving at the KpnI restriction endonuclease site at position 2325 in the sequence shown in Formula I. Plasmid pCIB10/35SBt (FIG. 14) is digested with BamHI and KpnI, and the approximately 2.2-kbp BamHI/KpnI fragment containing the deleted protoxin gene is isolated by preparative agarose gel electrophoresis. To convert the KpnI site at the ₃′ end to a BamHI site, the fragment is mixed with a KpnI/BamHI adapter oligonucleotide and ligated. This fragment is then mixed with BamHI-cleaved pCIB10/710 (FIG. 13). The resulting transformants, designed pCIB10/35SBt(KpnI) and shown in FIG. 15, contain the deleted protoxin gene of approximately 725 amino acids. These transformants are selected on kanamycin.

### Example 8

### Construction of a deleted Bacillus Thuringiensis protoxin gene containing approximately 645 amino acids, and construction of a chimeric gene containing this deleted gene with the CaMV 35S promoter

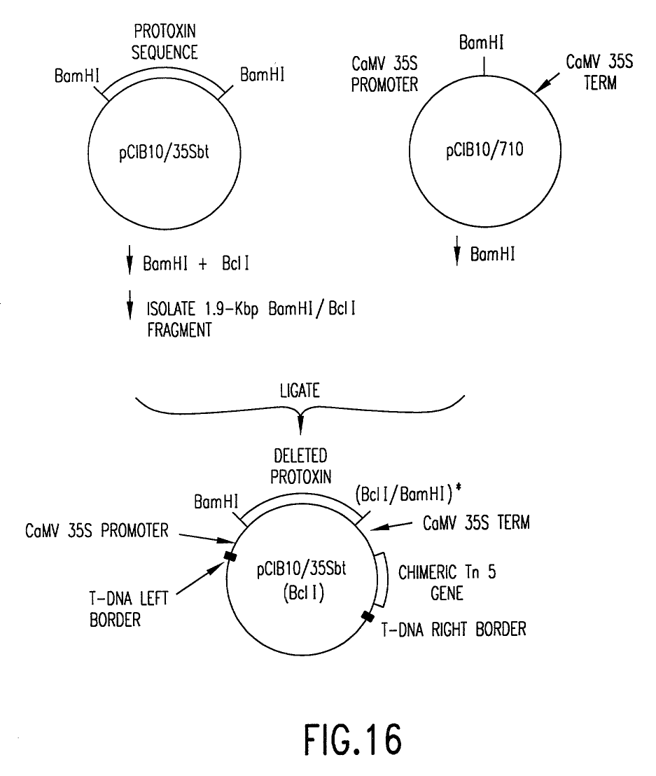

A deleted protoxin gene containing approximately 645 amino acids is made by removing the COOH-terminal portion of the gene by cleaving at the BclI restriction endonuclease site at position 2090 in the sequence shown in Formula I. Plasmid pCIB10/35SBt (FIG. 14) is digested with BamBI and BclI, and the approximately 1.9-kbp BamHI/BclI fragment containing the deleted protoxin gene is isolated by preparative agarose gel electrophoresis. Since BclI creates a cohesive end compatible with BamHI, no further manipulation is required prior to ligating this fragment into BamHI-cleaved pCIB10/710 (FIG. 13). The resulting plasmid, which has the structure pCIB10/35SBt(BclI) shown in FIG. 16, is selected on kanamycin.

### Example 9

### Construction of a deleted Bacillus thuringiensis protoxin gene containing approximately 607 amino acids, and construction of a chimeric gene containing this deleted gene with the CaMV 35S promoter

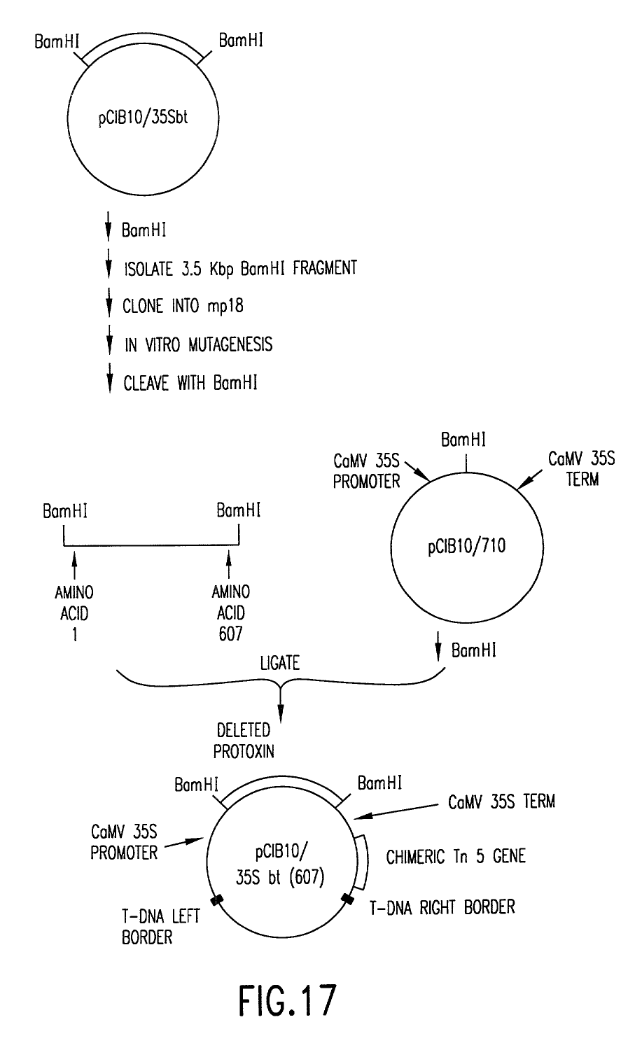

A deleted protoxin gene is made by introducing a BamHI cleavage site (GGATCC) following nucleotide 1976 in the sequence shown in Formula I. This is done by cloning the BamHI fragment containing the protoxin sequence from pCIB10/35SBt into mp18, and using standard oligonucleotide mutagenesis procedures described above. After mutagenesis, double-standard replicative form DNA is prepared from the M13 clone, which is then digested with BamHI. The approximately 1.9-kbp fragment containing the deleted protoxin gene is inserted into BamHI-cleaved pCIB10/710. The resulting plasmid, which the structure pCIB10/3SSBt(607) shown in FIG. 17, is selected for on kanamycin.

Some of the following Examples describe specific protocols for transforming cotton cells and regenerating cotton plants from cotton cells and callus. It should be understood that those with ordinary skill in the art may vary the details of the protocols while still remaining within the limits of the present invention. For example, numerous plant tissue culture media are know, some of which are described in detail below. The ordinarily skilled tissue culture scientist would know how to vary these solutions in order to achieve the same or similar results. Thus, Example 11 discloses a modified White's stock solution as a seed germination and callus development media; Example 12 describes a murashige and Skoog stock solution as a callus growth/maintenance media; Example 14 describes a Beasley and Ting stock solution as a plant germination medium. The ordinarily skilled tissue culture scientist knows how to vary these solutions in order to achieve results similar to those described in the Examples. Thus, the sugar in the callus growth medium may be glucose, which minimizes phenolic secretions, or sucrose, which promotes the formation of embryogenic callus.

The explants used in the transformation procedure may be from any suitable source, such as from seedlings, especially a hypocotyl or cotyledon, or from immature embryos of developing fruit.

Any antibiotic toxic to Agrobacterium may be used to kill residual Agrobacterium after the transformation step. Cefotaxime is preferred.

10. Regeneration of cotton plants (from Biner, Ciba-Geigy U.S. application, filed on the same day as the present application).

a) media

All media in this example contain Murashige and Skoog inorganic salts and Gamborg's B-5 vitamins, are adjusted to pH 5.7, and have the following composition (mg/l):

Macronutrients

| | |
|---|---|
| MgSO₄.7H₂O | 370 |
| KH₂PO₄ | 170 |
| KNO₃ | 1900 |
| NH₄NO₃ | 1650 |
| CaCl₂.2H₂O | 440 |

Micronutrients

| | |
|---|---|
| H₃BO₃ | 6.2 |
| MnSO₄.H₂O | 15.6 |
| ZnSO₄.7H₂O | 8.6 |
| NaMoO₄.2H₂O | 0.25 |
| CuSo₄.5H₂O | 0.025 |
| CaCl₂.6H₂O | 0.025 |
| KI | 0.83 |
| FeSO₄.7H₂O | 27.8 |
| Na₂EDTA | 37.3 |

Vitamins

| | |
|---|---|
| Thiamine.HCl | 10 |
| Pyridoxine.HCl | 1 |
| Nicotinic acid | 1 |
| Myo-Inositol | 100 |

In addition, the various media have the following components.

| Medium # | Additional Components |
|---|---|
| 1 | 20 g/l sucrose, 0.6% noble agar (Difco) |
| 2 | 30 g/l glucose, 2 mg/l |
| | alpha-naphthaleneacetic acid |
| | 1 mg/l kinetin, 0.8% noble agar |
| 3 | 30 g/l sucrose, 2 mg/l |
| | alpha-naphthaleneacetic acid |
| | 1 mg/l kinetin, 0.8% noble agar |
| 4 | 20 g/l sucrose, 0.5 mg/l picloram |
| 5 | 20 g/l sucrose, 5 mg/l |
| | 2,4-dichlorophenoxyacetic acid |
| 6 | 20 g/l sucrose, 15 mM glutamine |

Media at 25° C., 28° C. and 31° C. refer, in addition to the temperature, to a photoperiod of 16 hours light: 8 hours dark at a light intensity of 20 microEm⁻²_{ₛ}⁻².

b) Seed Sterilization and Planting

Seeds of cotton (Gossypium hirsutum var. Coker 310) are delinted by placing seed in concentrated H₂SO₄ for 2 min. Seeds are then washed 4 times with sterile, distilled water, dipped in 95% ethanol, flamed and planted on Medium #1 at 31° C.

c) Callus induction

Seven days following planting, seedling hypocotyls are excised, sliced longitudinally, cut into 2 mm sections and placed on Medium #2 at 31° C. Hypocotyl sections (2 mm) are transferred weekly to fresh Medium #2 and these cultures are also maintained at 31° C. Following 4 weekly transfers to Medium #2, callus tissue proliferating on the hypocotyl sections is removed from the original explant and placed on Medium #3 at 31° C. The callus is transferred to fresh Medium #3 after one month and maintained for an additional 1 to 2 months.

d) Suspension Culture Initiation

For initiation of suspension cultures, 100 mg of callus tissue is placed into 35 ml of Medium #4 in a 125 ml DeLong flask. Suspensions are rotated for 6 weeks at 140 rpm, and 28° C., at which time they begin rapidly to proliferate.

e) Embryo Development and Plant Regeneration

The embryos that form in Medium #4 proliferate even faster following replacement of Medium #4 by Medium #5. This embryogenic suspension is divided and subcultured every 3-7 days into fresh Medium #5. For development of embryos proliferating in Medium #5, the embryos are washed with, and then placed into, Medium #6. Three to four weeks following transfer to Medium #6, the mature embryos are placed on a solid medium at 25° C. The solid medium consists of a modified MS medium containing MS salts with 40 mM KNO₃ in place of KNO₃ and NH₄NO₃, B-5 vitamins, 2% sucrose, 15 mM glutamine, and solidified with 0.2% Gelrite (pH 5.7). Embryos are placed in petri dishes at 25° C. Shoot development is sporadic on this medium and root elongation is enhanced with the transfer of the embryos to the above modified MS medium without glutamine. Germinating embryos are then planted in vermiculite in 4″ pots and covered with a beaker (25° C.). After plantlets are established in vermiculite, the beaker is removed. Following one week at 28° C., the plantlets are placed in the greenhouse for further development into plants.

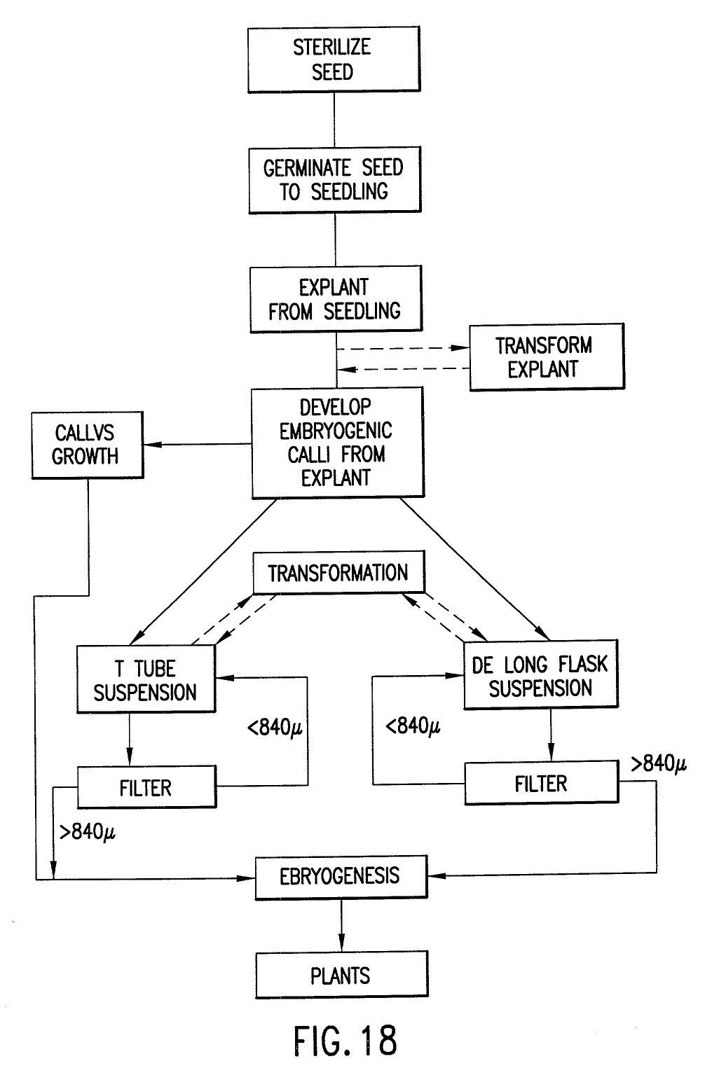

11-28. Regeneration of Cotton Plants (substantially from Phytogen U.S. Application, filed on same day as present application; see FIG. 18).

SEED GERMINATION AND CALLUS DEVELOPMENT MEDIA
COMPOSITION OF MODIFIED WHITE'S STOCK SOLUTION
(Phytomorphology 11:109-127, 1961)
(incorporated herein by reference)

| | | |
|---|---|---|
| | Concentration | |
| Component | per 1000 ml. | Comments |

| | | | |
|---|---|---|---|
| MgSO₄.7H₂O | 3.6 | g | Dissolve and make up |
| Na₂SO₄ | 2.0 | g | the final volume to |
| NaH₂PO₄.H₂O | 1.65 | g | 1000 ml. Label White's |
| | | | A Stock. Use 100 ml/l |
| | | | of final medium. |
| Ca(NO₃)₂.4H₂O | 2.6 | g | Dissolve and make up |
| KNO₃ | 800 | mg | the final volume to |
| KCl | 650 | mg | 1000 ml. Label White's |
| | | | B Stock. Use 100 ml/l |
| | | | of final medium. |
| Na₂MoO₄.2H₂O | 2.5 | mg | Dissolve and make up |
| CoCl₂.6H₂O | 2.5 | mg | the final volume to 100 |
| MnSO₄.H₂O | 300 | mg | ml. Label White's C |
| ZnSO₄.7H₂O | 50 | mg | Stock. Use 1.0 ml/l of |
| CuSO₄.5H₂O | 2.5 | mg | final medium. |
| H₃BO₃ | 50 | mg | |
| Fe-EDTA | | | Use 10 ml/l of MSFe |
| | | | EDTA. (See below) |
| Organic | | | Use 10 ml/l of MS |
| | | | organic. (See below) |

CALLUS GROWTH/MAINTENANCE MEDIA
COMPOSITION OF MURASHIGA & SKOOG (MS)
STOCK SOLUTIONS
(Physiol. Plant 15:473-497, 1962)
(incorporated herein by reference)

| | | |
|---|---|---|
| | Concentration per | |
| Component | 1000 ml. of Stock | Comments |

| | | | |
|---|---|---|---|
| NH₄NO₃ | 41.26 | g | Dissolve and make up |
| KNO₃ | 47.50 | g | the final volume to |
| CaCl₂.2H₂O | 11.00 | g | 1000 ml. Label MS-Major |
| MgSO₄.7H₂O | 9.25 | g | Use 40 ml/l of final |
| KH₂PO₄ | 4.25 | g | medium. |
| KI | 83 | mg | Dissolve and make up |
| H₃BO₃ | 620 | mg | the final volume to |
| MnSO₄.H₂O | 1690 | mg | 1000 ml. Label MS- |
| ZnSO₄.7H₂O | 860 | mg | Minor. Use 100 ml/l |
| Na₂MoO₄.2H₂O | 25 | mg | of final medium. |
| CuSO₄.5H₂O | 2.5 | mg | |
| CoCl₂.6H₂O | 2.5 | mg | |
| Nicotinic acid | 50 | mg | Dissolve and make up |
| Pyridoxin HCl | 50 | mg | the final volume to |
| Thiamine HCl | 10 | mg | 1000 ml. Label MS- |
| | | | Organic. Freeze in 10 |
| | | | ml aliquots. Use 10 |
| | | | ml/l of final medium. |
| Fe SO₄.7H₂O | 2.78 | g | Dissolve 2.78 g of |
| Na₂ EDTA.2H₂O | 3.73 | g | FeSO₄.7H₂O in about 200 |
| | | | ml of deionized water |
| | | | Dissolve 3.73 g of Na₂- |
| | | | EDTA.2H₂O (disodium |
| | | | salt of ethylenediamino- |
| | | | tetraacetic acid dihy- |
| | | | drate) in 200 ml of de- |
| | | | ionized water in another |
| | | | beaker.Heat the Na₂- |
| | | | EDTA solution on a hot |
| | | | plate for about 10 minutes. |
| | | | While constantly stirring, |
| | | | add FeSO₄ solution |
| | | | to Na₂-EDTA solution. |
| | | | Cool the solution to room |
| | | | temperature and make up |
| | | | the volume to 1000 ml. |
| | | | Label MSFe-EDTA. Cover |
| | | | bottle with foil and store |
| | | | in refrigerator. Use 10 ml/l |
| | | | of final medium. |
| Thiamine HCl | 50 | mg | Dissolve and make up the |
| | | | volume to 500 ml. Label |
| | | | MS-Thiamine. Use 4.0 ml/l |
| | | | of final medium. |
| m-Inositol | 10 | g | Dissolve and make up the |
| Glycine | 0.2 | g | final volume to 1000 ml |
| | | | Label MS-glycine/inositol. |
| | | | Use 10 ml/l of final medium. |

COMPOSITION OF BEASLEY AND TING'S STOCK SOLUTION
(Am. J. Bot. 60:130-139, 1973)

| | | |
|---|---|---|
| | Concentration | |
| Component | per 1000 ml. | Comments |

| | | | |
|---|---|---|---|
| KH₂PO₄ | 2.72 | g | Dissolve and make up |
| H₃BO₃ | 61.83 | mg | the volume to 100 ml. |
| Na₂MoO₄.2H₂O | 2.42 | mg | Label B&T-A Stock. |
| | | | Use 10 ml/l of final |
| | | | medium. |
| CaCl₂.2H₂O | 2.6 | g | Dissolve and make up |
| KI | 8.3 | mg | the volume to 100 ml. |
| CoCl₂.6H₂O | 0.24 | mg | Label B&T-B Stock. |
| | | | Use 10 ml/l of final |
| | | | medium. |
| MgSO₄.7H₂O | 4.93 | g | Dissolve and make up |
| MnSO₄.H₂O | 169.02 | mg | the volume to 100 ml. |
| ZnSO₄.7H₂O | 86.27 | mg | Label B&T-C Stock. |
| CuSO₄.5H₂O | 0.25 | mg | Use 10 ml/l of final |
| | | | medium. |
| KNO₃ | 25.275 | g | Dissolve and make up the |
| | | | volume to 200 ml. Label |
| | | | B&T-D Stock. Use 40 |
| | | | ml/l of final medium. |
| Nicotinic acid | 4.92 | mg | Dissolve and make up the |
| Pyridoxine HCl | 8.22 | mg | final volume to 100 ml. |
| Thiamine HCl | 13.49 | mg | Label B&T-Organics. Use |
| | | | 10 ml/l of final medium. |
| Fe-EDTA | | | Use 10 ml/l of MS-Fe- |
| | | | EDTA. |
| Inositol | | | 100 mg/l of final medium |
| NH₄NO₃ (15 μm) | | | 1200.6 mg/l of final |
| | | | medium. |

### Example 15

### Regeneration of plants starting from cotyledon explants

Seeds of Acala cotton variety SJ2 of Gossypium hirsutum are sterilized by contact with 245% alcohol for three minutes, then twice rinsed with sterile water and immersed with a 15% solution of sodium hypochlorite for 15 minutes, then rinsed in sterile water. Sterilized seeds are germinated on a basal agar medium in the darkfor approximately 14 days to produce a seedling. The cotyledons of the seedlings are cut into segments of which are transferred aseptically to a callus inducing medium [see above] consisting of Murashige and Skoog (MS) major and minor salts supplemented with 0.4 mg/l thiamine-HCl, 30 g/l glucose, 2.0 mg/l alpha-naphthaleneacetic acid (NAA), 1 mg/l kinetin, 100 mg/l of m-inositol, and agar (0.8%). The cultures are incubated at about 30° C. under conditions of 16 hours light and 22 hours darkness in a Percivall incubator with fluorescent lights (cool daylight) providing a light intensity of about 2000-4000 lux. Calli are formed on the cultured tissue segments within 3 to 4 weeks and are white to gray-greenish in color. The calli formed are subcultured every three to four weeks onto a callus growth medium comprising MS medium containing 100 mg/l m-inositol, 2.0 g/l sucrose, 2 mg/l alpha-naphthalenacetic acid (NAA) and agar. Somatic embryos formed four to six months after first placing tissue explants callus inducing medium. The callus and embryos are maintained on callus growth medium by subculturing onto fresh callus growth medium every three to four weeks.

Somatic embryos which formed on tissue pieces are explanted either to fresh callus growth medium, or to Beasley & Ting's medium (embryo germination medium). The somatic plantlets which are formed from somatic embryos are transferred onto Beasley and Ting's medium which contained 15 mg/l ammonium nitrate and 15 mg/l casein hydrolysate as an organic nitrogen source. The medium is solidified by a solidifying agent (Gelrite) and plantlets are placed in Magenta boxes. The somatic embryos developed into plantlets within about three months. The plantlets are rooted at the six to eight leaf stage [about three to four inches tall], and are transferred to soil and maintained in an incubator under high humidity for three to four weeks, after which they are transferred to the greenhouse. After hardening, plants are transferred to open tilled soil.

### Example 16

### Regeneration of plants starting from cotyledon explants-Variation 1.

The procedure of Example 15 is repeated using instead half-strength MS medium in which all medium components have been reduced to one-half the specified concentration. Essentially the same results are obtained.

### Example 17

### Regeneration of different cotton varieties from cotyledon explants.

The procedure of Examples 15 and 16 is repeated with Acala cotton varieties SJ4, SJ5, SJ2C-1, GC510, B1644, B2724, B1810, the picker variety Siokra and the stripper variety FC2017. All are successfully regenerated.

### Example 18

### Regeneration of cotton plants from cotyledon explants with suspension cell culture as intermediate step.

The procedure of Example 15 is repeated to the extent of obtaining callus capable of forming somatic embryos. Pieces of about 750-1000 mg of actively growing embryogenic callus is suspended in 22 ml units of liquid suspension culture medium comprised of MS major and minor salts, supplemented with 0.4 mg/l thiamine HCl, 20 g/l sucrose, 100 mg/l of inositol and alpha-naphthaleneacetic acid (2 mg/l) in T-tubes and placed on a roller drum rotating at 1.5A rpm under 16:8 light:dark regime. Light intensity of about 2000-4500 lux is again provided by fluorescent lights-(cool daylight). After four weeks, the suspension is filtered through an 840 micron size nylon mesh to remove larger cell clumps. The fraction smaller than 840 microns are allowed to settle, ished once with about 20-25 ml of fresh suspension culture medium. This cell suspension is transferred to T-tubes (2 ml per tube) and each tube diluted with 15 ml of fresh suspension culture medium. The cultures are maintained by repeating the above at 10-12 day intervals. At each subculture, the suspension is filtered and only the fraction containing cell aggregates smaller than 840 microns is transferred to fresh suspension culture medium. In all instances, the fraction containing cell clumps larger than 840 microns is placed onto the callus growth medium to obtain mature somatic embryos. The somatic embryos that are formed on callus growth medium are removed and transferred to embryo germination medium. Using the protocol of Example 6, these are germinated, developed into plantlets and then field grown plants.

### Example 19

### Regeneration of cotton plants from cotyledon explants with suspension cell culture as an intermediate step-Variant 1.

The procedure of Example 18 is repeated except that suspension cultures are formed by transferring 750-1000 mg of embryogenic calli to a DeLong flask containing 15-20 ml of the MS liquid medium containing 2 mg/l NAA. The culture containing flask is placed on gyrotory shaker and shaken at 100-110 strokes/minute. After three weeks the suspension is filtered through an 840 micron nylon mesh to remove the large cell clumps for plant growth, as in Example 18. The less than 840 micron suspension is allowed to settle, washed once in the MS liquid medium and resuspended in 2 to 5 ml of the MS liquid medium. The suspension is subcultured by transfer to fresh medium in a DeLong flask containing 1-2 ml of suspension and 15 ml of fresh MS liquid medium. The cultures are maintained by repeating this procedure at seven to ten day intervals. At each subculture only the less than 840 micron suspension is subcultured and the large clumps (840 microns or greater) are used for plant growth.

### Example 20

### Production of plants from large clumps of suspension cultured cells [example 19]

After three or four subcultures using the suspension growth procedure of Examples 18 and 19, 1.5 to 2.0 ml of cell suspension from the T-tube and DeLong flask are in each instance plated onto agar-solidified MS medium containing 2 mg/l NAA and Beasley & Ting medium containing 500 mg/l casein hydrolysate. Within three to four weeks embryogenic calli with developing embryos became visible. Again, the 840 micron or greater cell clumps are plated on the callus growth medium giving rise to embryogenic clumps with developing embryos which ultimately grew into plants.

### Example 21

### Transformation of Cotton Suspension Culture Cells to Tumorous-Phenotype by Agrobacteria LBA 4434.

a/ Growth of the plant suspension culture. An Acala cotton suspension culture is subcultured into “T” tubes with the medium (MS medium containing 2 g/liter NAA) being changed every seven to ten days. After a medium change, the “T” tube is rotated 240° and the cells allowed to settle out. The supernatant is removed by pipeting prior to transformation and the resulting cells treated as described below.

b/ Description of Agrobacterium vector. The Agrobacterium strain LBA 4434 (Hoekema, A. et al. Nature 303: 179-180, 1983) contains a Ti plasmid-derived binary plant transformation system. In such binary systems, one plasmid contains the T-DNA of a Ti-plasmid, the second plasmid contains the vir-region of a Ti-plasmid, and together the two plasmids function to effect plant (transformation. In the strain LBA 4434, the T-DNA plasmid pAL 1050 contains TL of pTiAch5, an octopine Ti-plasmid. The vir plasmid in strain LBA 4434, pAL 4404, contains the intact virulence regions of pTiAch 5 (Ooms, G. et al. Plasmid 21: 15-29, 1982). Strain LBA 4434 is available from Dr. Robert Schilperoort of the Department of Biochemistry, University of Leiden, The Netherlands.

c/ Growth of Agrobacteria. The transforming Agrobacterium strain is taken from a glycerol stock, inoculated in a small overnight culture, from which a 50-ml culture is inoculated the following day. Agrobacteria are grown on YEB medium [YEB is per liter in water: 5 g beef extract, 1 g yeast extract, 5 g peptone, 5 g sucrose, adjusted to pH 21.2 with NaOH. After autoclaving, 1 ml of 2 M MgCl₂ is added] to which antibiotics as appropriate have been added. The absorbance at 600 nm of the 50 ml overnight culture is read, the culture is centrifuged and the pellet resuspended in the plant cell growth medium (MS medium plus NAA at 2 mg/ml) to a final absorbance at 600 nm of 0.5. Eight ml of this bacterial suspension is added to each “T” tube containing the plant cells from part a above.

d/ Infection. The “T”tube containing the plant and bacteria cells is agitated to resuspend all cells and returned to a roller drum for three hours to allow the Agrobacteria to attach to the plant cells. The cells are then allowed to settle and the residual supernatant removed. A fresh aliquot of growth medium is added to the “T” tube and this allowed to incubate on a roller drum for a period of 18 to 20 hours in the presence of any residual Agrobacteria which remained. After this time, the cells are again allowed to settle, the supernatant is removed and the cells are ished twice with a solution of growth medium containing cefotaxime (200 ug/ml). After ishing, the cells from each T-tube are resuspended in 10 ml growth medium containing cefotaxime (200 ug/ml in all cases) and 1 ml aliquots of this plated on petri dishes.

e/ Growth of Transformed Tissue. The cells infected with Agrobacteria grew on the growth medium which had no added phytohormones, indicating the tissue had received the wild-type phytohormone genes in T-DNA.

These cells developed into tumors, further indicating transformation of the cultures.

### Example 22

### Cotton Suspension Culture Cells to a Kanamycin-resistant non-tumorous phenotype.

The same procedure as in Example 21 is followed except that different transforming Agrobacteria is used and that the plant selection medium contained an antibiotic for the selection of transformed plant tissue.

a/ Growth of Plant Tissue. As in Example 21, part a.

b/ Description of Agrobacterium. The transforming Agrobacteria contained the T-DNA containing binary vector pCIB 10 (Rothstein, S. J. etal. Gene 53: 153-161, 1987) as well as the pAL 4404 vir plasmid. The T-DNA of pCIB 10 contains a chimeric gene composed of the promoter from nopaline synthase, the coding region from Tn5 [encoding the enzyme neomycin phosphotransferase], and the terminator from nopaline synthase.

c/ Growth of Agrobacteria. Agrobacteria containing pCIB 10 are grown on YEB containing kanamycin (50 ug/ml). Otherwise, conditions are as in Example 21, part c.

d/ Infection. Transformation is accomplished as detailed in Example 21 with the change that the 1 ml aliquots resulting in part c are plated immediately on medium containing selective antibiotics. Selection medium contained either kanamycin (50 ug/ml) or G418 (25 ug/ml). Expression of the nos/neo/nos chimeric gene in transformed plant tissue allows the selection of this tissue on either of these antibiotics.

e/ Growth of Transformed Tissue. Plant growth media in this and all following examples contained phytohormones as indicated in Example 1.

In 2-4 weeks, transformed tissue became apparent on the selection plates. Uninfected tissue or control tissue showed no signs of growth, turned brown and died. Transformed tissue grew very well in the presence of kanamycin or G418.At this time, tissue pieces which are growing well are subcultured to fresh selection medium.

f/ Growth of Somatic Embryos. Somatic embryos formed on these tissue pieces. Somatic embryos are explanted to fresh medium (non selective).

g/ Germination. When the embryos had begun to differentiate and germinate, ie at the point where they are beginning to form roots and had two or three leaves, they are transferred to Magenta boxes containing growth medium. Growth is allowed to proceed until the plantlet had 15 to 22-leaves, at which time it is removed from the agar medium.

h/ Growth of Plantlet. The plantlet is now placed in potting soil, covered with a beaker to maintain humidity and placed in a Percival incubator for 4 - 22 weeks. At this time, the beaker is removed and the plant is transfered to the greenhouse.

i/ Growth of Plant in Greenhouse. The plants grew in the greenhouse, floared and set seed.

NO EXAMPLE 23 INCLUDED.

### Example 24

### Transformation of Cotton Suspension Culture Cells to a Hygromycin-resistant non-tumorous phenotype.

The same procedure as in Example 22 is followed except where noted. Different transforming Agrobacteria are used and the plant selection medium contained an antibiotic appropriate for the selection of transformed plant tissue.

b/ Description of Agrobacterium. The transforming Agrobacteria contained the T-DNA containing binary vector pCIB 2115 (Rothstein, S. J. etal. Gene 53: 153-161, 1987) as well as the vir plasmid. The T-DNA of pCIB 2115 contains a chimeric gene composed of the promoter and terminator from the cauliflower mosaic virus (CaMV) 35S transcript [Odell et al, Nature 313: 2210-812, 1985] and the coding sequence for hygromycin B phosphotransferase [Gritz, L. and J. Davies, Gene 25: 179-188).

c/ Growth of Agrobacteria. Agrobacteria containing pCIB 2115 are grown on YEB containing kanamycin (50 ug/ml).

d/ Infection. Transformation is accomplished as detailed in Example 21 with the change that the 1 ml aliquots resulting in part c are plated immediately on medium containing selective antibiotics. Selection medium contained 50 ug/ml hygromycin. Expression of the chimeric hygromycin gene in transformed plant tissue allows the selection of this tissue on medium containing hygromycin.

e/ Growth of Transformed Tissue. As in Example 22, part e except that the antibiotic hygromycin is used in the plant selection growth medium.

### Example 25

### Plant Extraction Procedure

Plant tissue is homogenized in Extraction Buffer [ca 100 mg in 0.1 ml Extraction Buffer].

### Leaf Extraction Buffer

50 mM Na₂CO₃ pH 9.5

10 mM EDTA

0.05% Triton X-100

0.05% Tween

100 mM NaCl

1 mM PMSF [Phenylmethylsulfonyl Fluoride] (add just prior to use)

1 mM leupeptine (add just prior to use).

After extraction, 2 M Tris pH 7.0 is added to adjust the pH of the extract to a pH of 8.0-8.5. The extract is then centrifuged 10 minutes in a Beckman microfuge and the supernatant used for ELISA analysis.

### Example 26

### ELISA Analysis of Plant Tissue

ELISA [enzyme-linked immunosorbent assay] are very sensitive, specific assays for antigenic material. ELISA assays are very useful for studying the expression of polypeptide gene products. The development of ELISA techniques as a general tool is described by M. F. Clark et al in Methods in Enzymology 118:742-766 (1986); this is herein incorporated by reference.

An ELISA for the Bt toxin was developed using standard procedures and used to analyze transgenic plant material for expression of Bt sequences. The steps used in this procedure are as given below:

1. ELISA plate is pretreated with ethanol.

2. Affinity-purified rabbit anti-Bt antiserum (50 ul) at a concentration of 3 ug/ml in borate-buffered saline [see below] is added to the plate and this allowed to incubate overnight at 4 degrees C. Antiserum was produced in response to immunizing rabbits with gradient-purified Bt crystals [Ang, B. J. & Nickerson, K. W.; Appl. Environ. Microbiol. 36: 625-626 (1978)) solubilized with sodium dodecyl sulfate.

3. Wash with ELISA Wash Buffer [see below].

4. Treat 1 hour at room temperature with Blocking Buffer [see below].

5. Wash with ELISA Wash Buffer [see below].

6. Add plant extract in an amount to give 50 ug of protein (this is typically ca. 5 microliters of extract). Leaf extraction buffer is described below; protein is determined by the Bradford method (Bradford, M., Anal. Biochem. 72: 248 (1976)] using a commercially available kit (Bio-Rad, Richmond, Calif.]. If dilution of the leaf extract is necessary, ELISA Diluent [see below] is used.

Allow this is incubate overnight at 4 degrees C.

7. Wash with ELISA Wash Buffer [see below].

8. Add 50 ul affinity-purified goat anti-Bt antiserum at a concentration of 3 ug/ml protein in ELISA Diluent [see below]. Allow this to incubate for one hour at 37 degrees C.

9. Wash with ELISA Wash Buffer [see below].

10. Add 50 ul rabbit anti-goat antibody bound to alkaline phosphatase [commercially available from Sigma Chemicals, St. Louis, Mo.]. This is diluted 1:500 in Diluent. Allow to incubate for one hour at 37 degrees C.

11.Wash with ELISA Wash Buffer [see below].

12.Add 50 microliters substrate [0.6 mg/ml p-nitrophenyl phosphate in ELISA Substrate Buffer (see below)].

Incubate for 30 minutes at room temperature.

13.Terminate reaction by adding 50 microliters of 3 M NaOH.

14.Read absorbance at 405 nm in modified ELISA reader [Hewlett Packard, Stanford, Calif.].

Plant tissue transformed with the pCIB10/35SBt(BclI) [see FIG. 16] construction, when assayed using this ELISA procedure shows a positive reaction, indicating expression of the Bt gene.

### Example 27

### ELISA BUFFERS

(ELISA Phosphate Buffered Saline)

| | |
|---|---|
| 10 mM NaPhosphate: | 4.68 grams/4 liters. |
| Na₂HPO₄ | NaH₂PO₄.H2O |
| 0.976 grams/4 liters | |
| 140 mM NaCl | 32.7 grams/4 liters |
| NaCl | |

pH should be approximately 7.4

Borate Buffered Saline

100 mM Boric acid

25 mM Na Borate

75 mM NaCl

Adjust pH to 8.4-8.5 with HCl or NaOH as needed.

ELISA Blocking Buffer

In PBS,

1% BSA

0.02% Na azide

ELISA Wash Buffer p0 10 mM Tris-HCl pH 8.0

0.05% Tween 20

0.02% Na Azide

2.5M TRIS

ELISA Diluent

In EPBS:

0.05% Tween 20

1% BSA

0.02% Na Azide

ELISA Substrate Buffer

In 500 mls,

48 ml Diethanolamine,

24.5 mg MgCl₂;

adjust to pH 9.8 with HCl.

ELISA Substrate

15 mg p-nitrophenyl phosphate in 25 ml Substrate Buffer

### Example 28

### Transformation of Cotton Cells to Insect Resistance and Bioassay of Transformed Cotton Cells

Transformed embryogenic cotton cultures are obtained as in Example 24, with the variation that the vector used also contains the delected Bacillus thuringiensis protoxin gene with the CaMV 35S promoter described in Example 8.

Following hygromycin selection, antibiotic-resistant embryos and cell clumps are individually transferred to separate petri plates with fresh callus growth medium containing hygromycin (100 mg/L) and allowed to grow until the fresh weight of the culture is between 0.1 g and 0.5 g. At this point, each culture is a mixture of embryogenic cells, cell clumps, and embryos and is judged to be transformed based on the demonstrated phenotype of hygromycin resistance. Each culture is subdivided in half. One half is maintained on callus growth medium lacking hygromycin, with the variation that the other part is maintained on medium that also contains hygromycin (100 mg/L). After a three week period of growth, the culture maintained in the absence of hygromycin selection is then used for the feeding bioassay. The following example gives the data from such an assay.

Heliothis virescens eggs laid on sheets of cheesecloth are obtained from the CIBA-GEIGY Corporation in Vero Beach, Fla. The cheesecloth sheets are transferred to a large covered glass beaker and incubated at 29 degrees C. with wet paper towels to maintain humidity. The eggs hatch within three days. As soon as possible after hatching, the larvae are transferred to the transformed embryogenic cotton cultures. Usually six (6) larvae are placed on each culture. Controls are cotton callus cultures which are not transformed. The number and size of the larvae are scored after a six day period of growth by grouping the larvae into the following classes: class 0=larvae dead; class 1=larvae 0-5 mm in length; class 2=larvae 5-10 mm in length; class 3=larvae 10-15 mm in length. Four control and 23 transformed plant cultures are assayed.

The data obtained are as follows:

| | |
|---|---|
| # larvae | % in class |

| Sample | Scored | 0 | 1 | 2 | 3 |
|---|---|---|---|---|---|
| control | 23 | 4% | | | 96% |
| transformed | 106 | 54% | 6% | 23% | 17% |

In addition to the increased mortality and smaller size of the Heliothis larvae placed on transformed cotton callus relative to control callus, the feeding behavior of the larvae on transformed samples is also different from the controls. Larvae on the transformed callus often stopped feeding and left the callus entirely.

Plant cultures for which no larvae feeding on them reached 10 mm in length are judged to have significant insecticidal activity compared to that of the controls. The portion of those cultures maintained separately on hygromycin selection was then plated and germinated to produce insecticidal plants according to the procedure of Example 24.

### Example 29

### Construction of pCIB1300,for high level expression in plants.

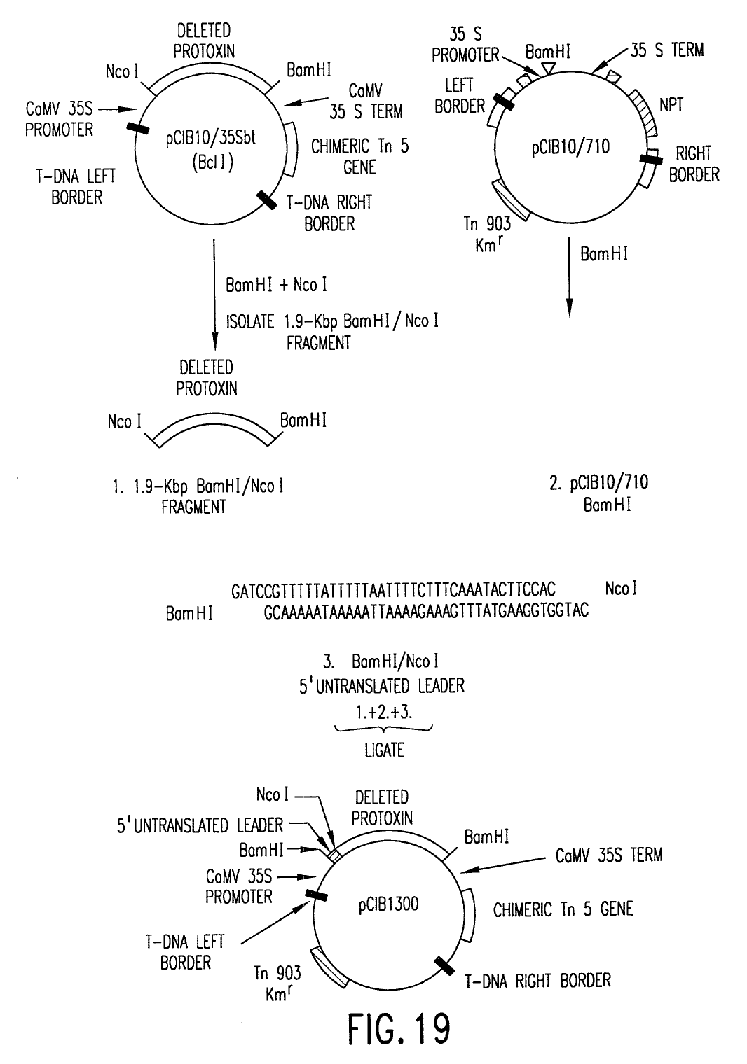

pCIB1300 is engineered for high level expression of the Bt gene and contains an untranslated leader sequence 5′ to the Bt gene to enhance expression in plants. The untranslated leader is a 40 bp sequence 5′ to the initiation codon of the Bt gene and 3′ to the CaMV 35S untranslated leader. The final pCIB1300 construct is engineered by the insertion of the 40 bp leader and deleted Bt gene into the BamHl site of pCIB10/710 as shown in FIG. 19. A 1.9 kb NcoI-BamHl fragment from pCIB10/35S Bt(Bcl)deletion is purified in low-gelling temperature agarose. The 40 bp leader is chemically synthesized as a double-stranded oligonucleotide with a 5′ overhanging BamHl site and a 3′ overhanging Ncol site using an Applied Biosystems DNA Synthesizer. The sequence of the untranslated leader as shown in the center of FIG. 19 is derived from the alfalfa mosaic virus (AMV) coat protein untranslated leader described by Koper-Zwarthoff et al. [Koper-Zwarthoff,E. C., Lockard,R. E., Alzner-Deweerd,B., RajBhandary,U. L. and J. F. Bol (1977) Proc. Natl. Acad. Sci. USA 74: 5504-5508]. The 40 bp leader, 1.9 kb Bt fragment and BamHl linearized pCIB710 vector are joined in a three-part ligation using T4 DNA ligase to construct pCIB1300.

### Example 30

### Isolation of cDNA clones coding for the small subunit of RuBPCase in Cotton

Gossypium hirsutum (Funk line RF522) plants are grown from seeds in the greenhouse with 14 hour daily light periods. Total RNA is isolated from young green leaves following the procedure of Newbury and Possingham (Plant Physiology 1977, 60: 543). PolyA⁺ RNA is purified as described in Maniatis et al. 1982 p.197. Double-stranded cDNA (complementary DNA) is synthesized according to the procedure of Okayama and Berg (Mol. Cell. Biol. 1982, 2:161) with the following modifications:

A. First strand cDNA is primed with oligo-dT;

B. After tailing the double-stranded cDNA with oligo-dG using polynucleotidyl-transferase, it is cloned into oligo-dC tailed pUC9 (Pst I site - from Pharmacia), and annealed; and

C. The DNA is transformed into E. coli strain HB101.

Since, with the chlorophyll a/b (Cab) binding protein, RuBPCase is the most abundant protein in green leaves, we then screened our cDNA library for cDNA clones of the most abundant mRNAs. Nitrocellulose (Schleicher and Schuell) filter replicas of the cDNA clones are screened with the first cDNA strand, radioactively labeled with alpha-dCT³²P and reverse-transcriptase, the template being the same polyA⁺ RNA as that used to construct the cDNA library. Six cDNA clones out of 275, are selected and analyzed further.

Northern analysis (done as described in Maniatis et al. 1982, p. 202) shows that two of these cDNA clones hybridize to a class of mRNA about 1100 nt long. They cross-hybridize with a Cab probe from tobacco. The other four hybridize to a class of mRNA 900 to 1000 nt long, a size consistent with that of the rbcs (small subunit of Rubisco). Cotton leaf mRNA, after hybrid selection using one of these four cDNA clones, is released and translated in vitro (as described in maniatis et al. 1982, p.329) using rabbit reticulocytes in vitro translation kit (Promega Biotec). Electrophoresis on polyacrylamide gel of the translation products showed one major polypeptide of about 20 Kd, a molecular weight consistent with that of the precursor of the rbcS. The other 3 cDNA clones cross-hybridize with the clone used for the hybrid-release experiment.

Large portions of these cDNA clones are sequenced, using the dideoxy chain-termination technique (Sanger et al., 1977) after subcloning into M13. Comparison of their sequences with formerly published rbcS sequences from other species showed that they are indeed rbcS cDNA clones.

### Example 31

### Isolation of genomic clones of small subunit RuBPcase of cotton

A. Cotton Genomic Southern analysis.

Genomic Southern blots are prepared by standard procedures using nitrocellulose filters. Prehybridization, hybridization and washing conditions are as described in Klessig et al. (Plant Mol. Biol. Reporter, 1983, 1:12). Genomic Southern analysis, using our rbcS cDNA clone as a probe, revealed 4 to 5 genomic fragments depending on the restriction enzyme used to digest the DNA. As expected, the rbcS is encoded by a small gene family in cotton, as in other species previously studied by others. The cotton rbcs multigene family is estimated to contain at least 5 members.

B. Isolation of rbcS genomic clones

In order to construct a cotton genomic library, partial Sau3a digests of cotton genomic DNA are size-fractionated on a 10% to 40% sucrose-gradient, and ligated into Lambda EMBL3 arms (Stratagene) digested with Bam HI. Packaging of Lambda recombinants, done using Packagene kit (Stratagene), is followed by transfection into E. coli strain K802. Nitrocellulose filter duplicate replicas are screened as described in Maniatis et al. 1982 p.320, using our rbcs cDNA clone from above as a probe. Twelve positive clones out of 450,000 plaques are purified. DNA is isolated from plate lysates of these recombinants phages, as described in Maniatis et al. (1982, p.80).

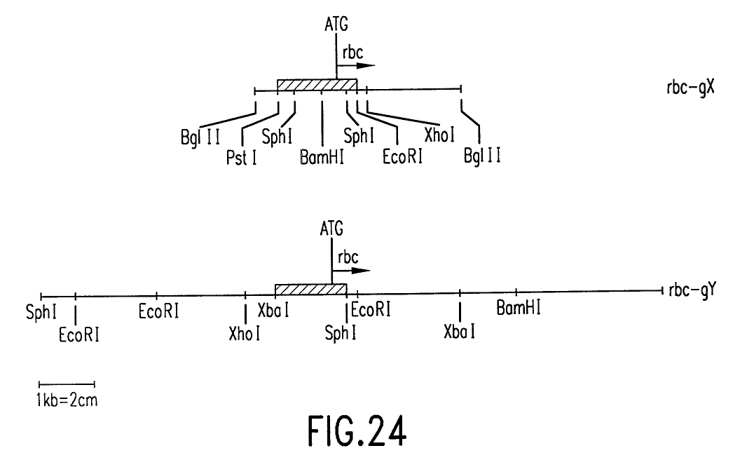

After comparing these genomic clones by their restriction digest pattern with various enzymes, five different rbcS genes are identified. Each one is subcloned into the plasmid vector pBSM13+ (Stratagene). These subclones are then mapped and partially sequenced in order to localize the 5′ end of the gene and the first ATG (translational start site) of the transit peptide. A map of two of these genomic subclones, rbc-gx and rbc-gY is shown on FIG. 24. The Lambda EMBL3 phages containing the genomic DNA of subclones rbc-gx and rbc-gY have been deposited with the American Type Culture Collection.

C. Study of the level of expression of the rbcs genes in cotton leaves

Forty-one additional rbcS cDNA clones are isolated from the cotton leaf cDNA library. Restriction mapping analysis, sequencing and hybrization of these cDNA clones to gene specific probes allows us to conclude that the gene carried by the genomic clone rbc-gX is responsible for about 17% of the leaf rbcS transcripts.

### Example 32

### Construction of chimeric genes using cotton rbcs promoter.

A. Insertion of an Nco I site at the first ATG of the rbcS transit peptides.

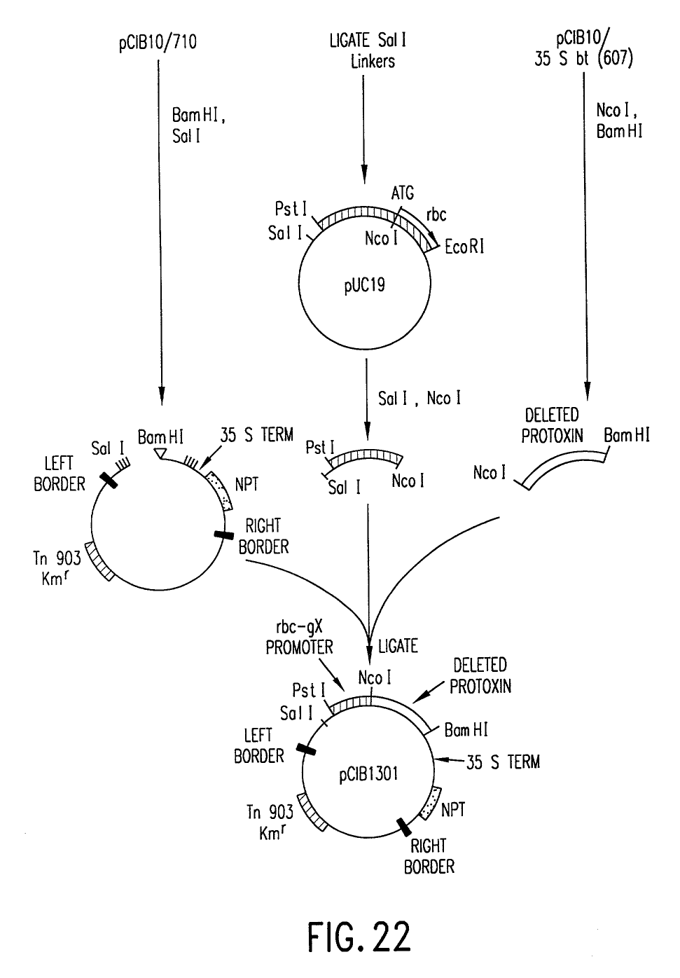

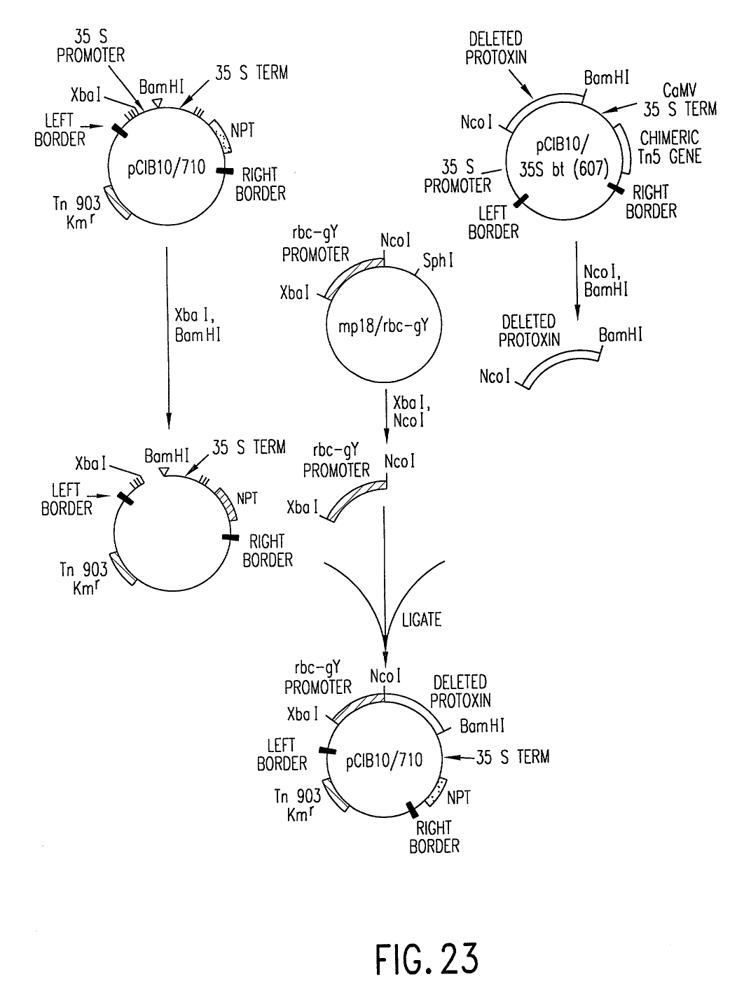

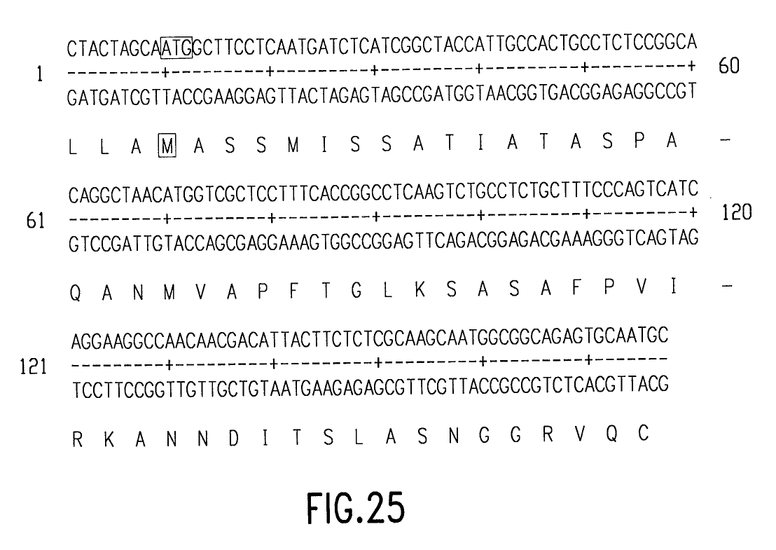

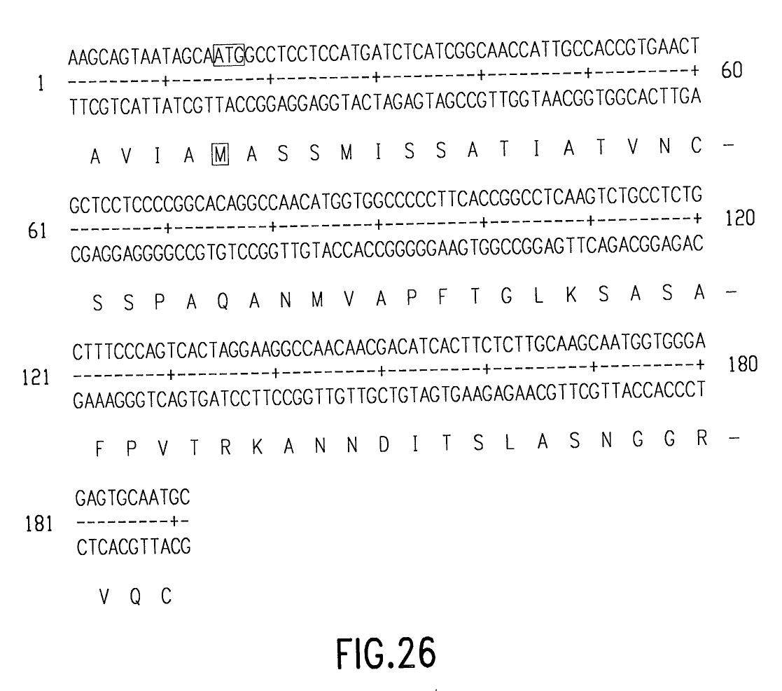

The sequences of the transit peptides of rbc-gx and rbc-gY are shown on FIGS. 26 and 25 respectively. An Nco I cleavage site (CCATGG) is introduced at the first ATG of the transit peptide of these two genes. This is done by cloning the PstI-EcoRI fragment of gene rbc-gx and the XbaI-SphI fragment of gene rbc-gY (hatched fragments on FIGS. 22 and 23 respectively) into mp18 and mp19 respectively, and using standard oligonucleotide site-directed mutagenesis procedures described above to introduce the NcoI site.

B. Construction of pCIB 1301, a plasmid bearing a chimeric gene containing the deleted Bacillus thuringiensis protoxin gene (607 deletion) with the rbc-gx gene promoter.

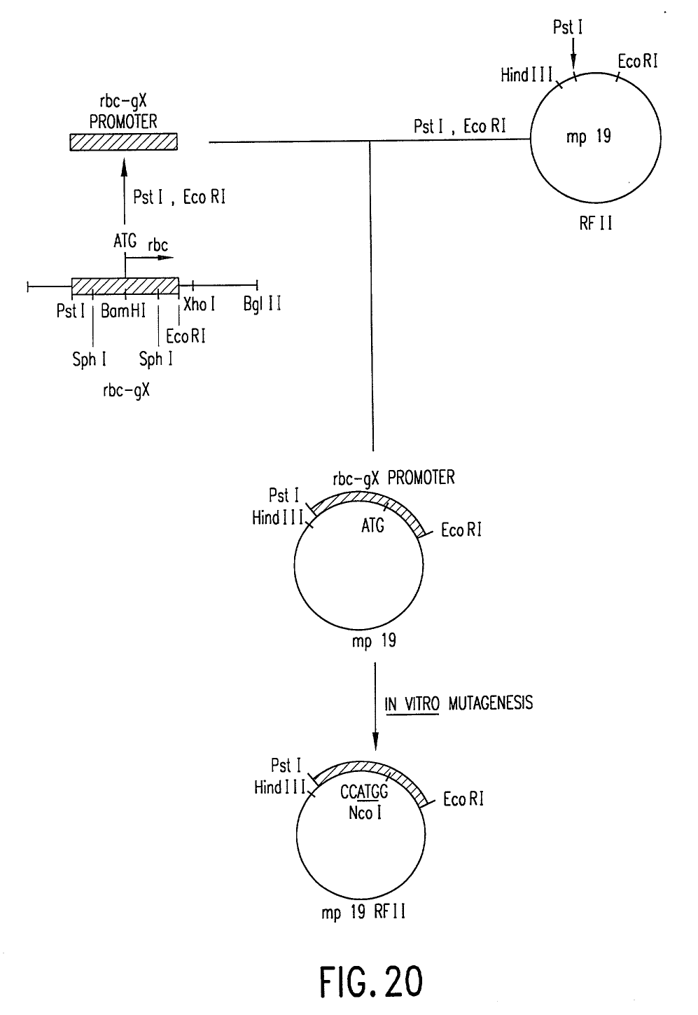

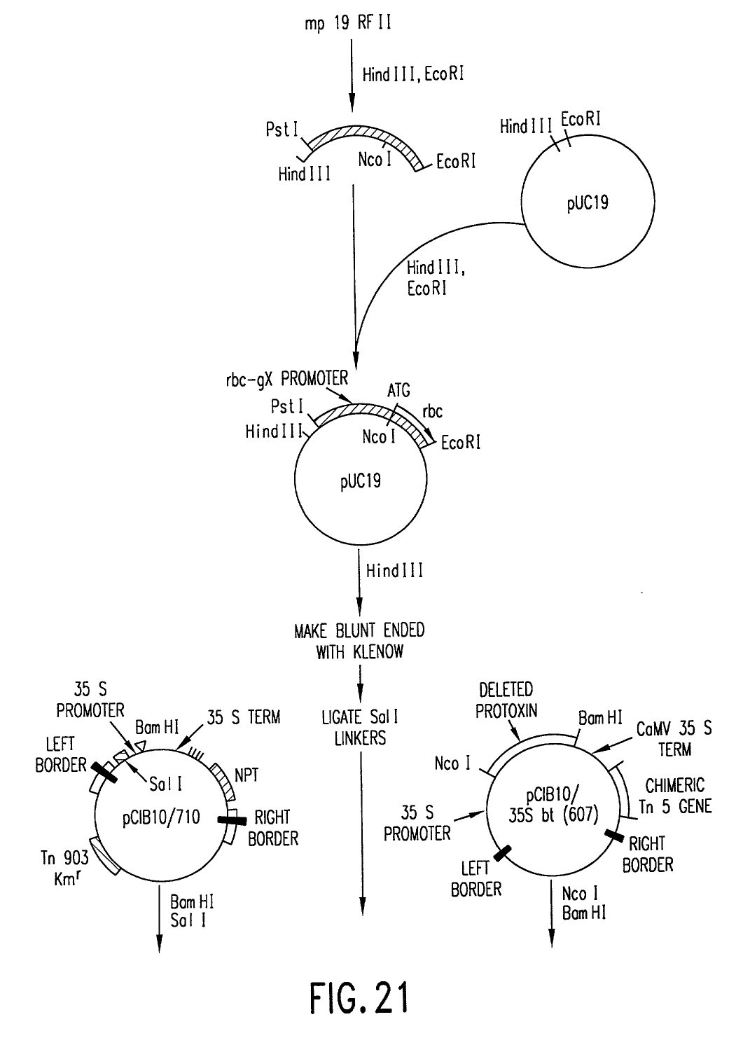

After the site-directed mutagenesis, double-stranded replicative form DNA is isolated from the M13 clone, which is then digested with Hind III and Eco RI. The Hind III-Eco RI fragment containing the rbc-gX promoter is ligated together with Hind III and Eco RI digested plasmid pUC19 and the ligation mix then transformed into E. coli strain HB101. Plasmid DNA is isolated from ampicillin-selected transformants and digested with HindIII. The ends of the resulting molecule are made blunt-ended by treatment with the Klenow subunit of DNA polymerase I and Sal I linkers are ligated to these ends. The resulting linear molecule is digested with Sal I and Nco I and gel-purified. In a three-part ligation the gel-purified Sal I-Nco I fragment is joined to a gel-purified Bam HI-Sal I fragment from pCIB770, a broad-host range replicon used as an Agrobacterium Ti plasmid cloning vector (Rothstein, et al.[1987] Gene 53: 153-164) and a gel-purified Nco I-Bam HI fragment containing the truncated 607 amino acid Bt gene. The ligation mix is transformed into E. coli strain HB101. The resulting plasmid, pCIB1301, which is depicted graphically in FIGS. 20, 21 and 22, is selected on kanamycin.

C. Construction of pCIB1302, a plasmid bearing a chimeric gene containing the deleted Bacillus thuringiensis protoxin gene (607 deletion) with the rbc-gY gene promoter.

After the mutagenesis, double-stranded replicative form DNA is isolated from the M13 clone, which is then digested with Xba I-Nco I. The approximately 1.97 kb Nco I-Bam HI fragment containing the deleted protoxin gene, is then ligated, together with the Xba I-Nco I rbc-gY promoter fragment, in a three way ligation, into Xba I-Bam HI cleaved pCIB10/710. The resulting plasmid, pCIB1302, the structure of which is shown in FIG. 23, is selected on kanamycin.

What we claim is:

1. A cotton cell comprising a chimeric gene that expresses a polypeptide having substantially the insect toxicity properties of Bacillus thuringiensis crystal protein.

2. The cell according to claim 1 wherein the plant cells are cells of Gossypium hirsutum, Gossypium arboreum, or Gossypium barbadense.

3. The cell according to claim 2 wherein the plant cells are cells of Gossypium hirsutum.

4. The cell according to claim 1 wherein the plant cells are of the variety-Acala SJ-2, Acala GC 510, Acala B-1644 or Siokra.

5. The cell according to claim 1 wherein the promoter, 5′ untranslated region, and, optionally, the 3′ untranslated region of the gene are derived from plant or plant virus genes.

6. The cell according to claim 5 wherein the promoter, 5′ untranslated region or 3 ′ untranslated region of the gene are derived from a plant gene that codes for the small subunit of ribose bisphosphate carboxylase or chlorophyll a/b-binding protein.

7. The cell according to claim 5 wherein the promoter, 5′ untranslated region or 3′ untranslated region are derived from a plant DNA virus.

8. The cell of claim 7 wherein the plant virus is cauliflower mosaic virus.

9. The cell of claim 8 wherein the cauliflower mosaic virus promoter is the 35S promoter of gene VI.

10. The cell of claim 1 wherein the promoter, 5′ untranslated region or the 3, untranslated region of the gene are derived from DNA sequences that are present in Agrobacterium plasmids, and that cause expression in plants.

11. The cell of claim 10 wherein the promoter is derived from the Ti plasmid of Agrobacterium tumefaciens.

12. The cell of claim 10 wherein the DNA sequences are derived from a gene that codes for octopine synthase.

13. The cell of claim 10 wherein the DNA sequences are derived from a gene that codes for nopaline synthase.

14. The cell of claim 1 wherein the polypeptide has an Mr of about 130,000 to about 140,000, or insecticidal fragments thereof.

15. The cell of claim 14 wherein the polypeptide is fused to another molecule.

16. The cell of claim 1 wherein the gene is substantially complementary to the nucleotide sequence that codes for the crystal protein delta endotoxin in B. thuringiensis.

17. The cell of claim 1 wherein the gene is capable of hydridizing to the coding region of the gene that codes for the crystal protein endotoxin in B. thuringiensis.

18. The cell of claim 14 wherein the polypeptide has substantially the same immunological properties as the crystal protein from Bacillus thuringiensis.

19. The cell of claim 16, 17 or 18 wherein a subspecies of Bacillus thruingiensis is kurstaki, berliner, alesti, tolworthi, sotto, and dendrolimus.

20. The cell of claim 19 wherein the Bacillus thuringiensis is the variety kurstaki HDl.

21. The cell of claim 20 wherein the gene express a polypeptide having the sequence given in FIG. II:
Formula II
MetAspAsnAsnProAsnIleAsnGluCysIleProTyrAsnCysLeuSerAsnProGlu - 20
ValGluValLeuGlyGlyGluArgIleGluThrGlyTyrThrProIleAspIleSerLeu -
SerLeuThrGlnPheLeuLeuSerGluPheValProGlyAlaGlyPheValLeuGlyLeu -
ValAspIleIleTrpGlyIlePheGlyProSerGlnTrpAspAlaPheLeuValGlnIle -
GluGlnLeuIleAsnGlnArgIleGluGluPheAlaArgAsnGlnAlaIleSerArgLeu -
GluGlyLeuSerAsnLeuTyrGlnIleTyrAlaGluSerPheArgGluTrpGluAlaAsp -
ProThrAsnProAlaLeuArgGluGluMetArgIleGlnPheAsnAspMetAsnSerAla -
LeuThrThrAlaIleProLeuPheAlaValGlnAsnTyrGlnValProLeuLeuSerVal -
TyrValGlnAlaAlaAsnLeuHisLeuSerValLeuArgAspValSerValPheGlyGln -
ArgTrpGlyPheAspAlaAlaThrIleAsnSerArgTyrAsnAspLeuThrArgLeuIle - 200
GlyAsnTyrThrAspHisAlaValArgTrpTyrAsnThrGlyLeuGluArgValTrpGly -
ProAspSerArgAspTrpIleArgTyrAsnGlnPheArgArgGluLeuThrLeuThrVal -
LeuAspIleValSerLeuPheProAsnTyrAspSerArgThrTyrProIleArgThrVal -
SerGlnLeuThrArgGluIleTyrThrAsnProValLeuGluAsnPheAspGlySerPhe -
ArgGlySerAlaGlnGlyIleGluGlySerIleArgSerProHisLeuMetAspIleLeu -
AsnSerIleThrIleTyrThrAspAlaHisArgGlyGluTyrTyrTrpSerGlyHisGln -
IleMetAlaSerProValGlyPheSerGlyProGluPheThrPheProLeuTyrGlyThr -
MetGlyAsnAlaAlaProGlnGlnArgIleValAlaGlnLeuGlyGlnGlyValTyrArg -
ThrLeuSerSerThrLeuTyrArgArgProPheAsnIleGlyIleAsnAsnGlnGlnLeu -
SerValLeuAspGlyThrGluPheAlaTyrGlyThrSerSerAsnLeuProSerAlaVal - 400
TyrArgLysSerGlyThrValAspSerLeuAspGluIleProProGlnAsnAsnAsnVal -
ProProArgGlnGlyPheSerHisArgLeuSerHisValSerMetPheArgSerGlyPhe -
SerAsnSerSerValSerIleIleArgAlaProMetPheSerTrpIleHisArgSerAla -
GluPheAsnAsnIleIleProSerSerGlnIleThrGlnIleProLeuThrLysSerThr -
AsnLeuGlySerGlyThrSerValValLysGlyProGlyPheThrGlyGlyAspIleLeu -
ArgArgThrSerProGlyGlnIleSerThrLeuArgValAsnIleThrAlaProLeuSer -
GlnArgTyrArgValArgIleArgTyrAlaSerThrThrAsnLeuGlnPheHisThrSer -
IleAspGlyArgProIleAsnGlnGlyAsnPheSerAlaThrMetSerSerGlySerAsn -
LeuGlnSerGlySerPheArgThrValGlyPheThrThrProPheAsnPheSerAsnGly - 580
SerSerValPheThrLeuSerAlaHisValPheAsnSerGlyAsnGluValTyrIleAsp - 600
ArgIleGluPheValProAlaGluValThrPheGluAlaGluTyrAspLeuGluArgAla -
GlnLysAlaValAsnGluLeuPheThrSerSerAsnGlnIleGlyLeuLysThrAspVal -
ThrAspTyrHisIleAspGlnValSerAsnLeuValGluCysLeuSerAspGluPheCys -
LeuAspGluLysLysGluLeuSerGluLysValLysHisAlaLysArgLeuSerAspGlu -
ArgAsnLeuLeuGlnAspProAsnPheArgGlyIleAsnArgGlnLeuAspArgGlyTrp -
ArgGlySerThrAspIleThrIleGlnGlyGlyAspAspValPheLysGluAsnTyrVal -
ThrLeuLeuGlyThrPheAspGluCysTyrProThrTyrLeuTyrGlnLysIleAspGlu -
SerLysLeuLysAlaTyrThrArgTyrGlnLeuArgGlyTyrIleGluAspSerGlnAsp -
LeuGluIleTyrLeuIleArgTyrAsnAlaLysHisGluThrValAsnValProGlyThr -
GlySerLeuTrpProLeuSerAlaProSerProIleGlyLysCysAlaHisHisSerHis - 800
HisPheSerLeuAspIleAspValGlyCysTyrAspLeuAsnGluAspLeuGlyValTrp -
ValIlePheLysIleLysThrGlnAspGlyHisAlaArgLeuGlyAsnLeuGluPheLeu -
GluGluLysProLeuValGlyGluAlaLeuAlaArgValLysArgAlaGluLysLysTrp -
ArgAspLysArgGluLysLeuGluTrpGluThrAsnIleValTyrLysGluAlaLysGlu -
SerValAspAlaLeuPheValAsnSerGlnTyrAspArgLeuGlnAlaAspThrAsnIle -
AlaMetIleHisAlaAlaAspLysArgValHisSerIleArgGluAlaTyrLeuProGlu -
LeuSerValIleProGlyValAsnAlaAlaIlePheGluGluLeuGluGlyArgIlePhe -
ThrAlaPheSerLeuTyrAspAlaArgAsnValIleLysAsnGlyAspPheAsnAsnGly -
LeuSerCysTrpAsnValLysGlyHisValAspValGluGluGlnAsnAsnHisArgSer -
ValLeuValValProGluTrpGluAlaGluValSerGlnGluValArgValCysProGly - 1000
ArgGlyTyrIleLeuArgValThrAlaTyrLysGluGlyTyrGlyGluGlyCysValThr -
IleHisGluIleGluAsnAsnThrAspGluLeuLysPheSerAsnCysValGluGluGlu -
ValTyrProAsnAsnThrValThrCysAsnAspTyrThrAlaThrGlnGluGluTyrGlu -
GlyThrTyrThrSerArgAsnArgGlyTyrAspGlyAlaTyrGluSerAsnSerSerVal -
ProAlaAspTyrAlaSerAlaTyrGluGluLysAlaTyrThrAspGlyArgArgAspAsn - 1100
ProCysGluSerAsnArgGlyTyrGlyAspTyrThrProLeuProAlaGlyTyrValThr -
LysGluLeuGluTyrPheProGluThrAspLysValTrpIleGluIleGlyGluThrGlu -
GlyThrPheIleValAspSerValGluLeuLeuLeuMetGluGluEnd -

22. The cell of claim 1 wherein the sequence of the coding region of the gene comprises the sequence of FIG. I:
Formula I

| | |
|---|---|
| 1 | GTTAACACCC TGGGTCAAAA ATTGATATTT AGTAAAATTA GTTGCACTTT |
| 51 | GTGCATTTTT TCATAAGATG AGTCATATGT TTTAAATTGT AGTAATGAAA |
| 101 | AACAGTATTA TATCATAATG AATTGGTATC TTAATAAAAG AGATGGAGGT |
| 151 | AACTTATGGA TAACAATCCG AACATCAATG AATGCATTCC TTATAATTGT |
| 201 | TTAAGTAACC CTGAAGTAGA AGTATTAGGT GGAGAAAGAA TAGAAACTGG |
| 251 | TTACACCCCA ATCGATATTT CCTTGTCGCT AACGCAATTT CTTTTGAGTG |
| 301 | AATTTGTTCC CGGTGCTGGA TTTGTGTTAG GACTAGTTGA TATAATATGG |
| 351 | GGAATTTTTG GTCCCTCTCA ATGGGACGCA TTTCTTGTAC AAATTGAACA |
| 401 | GTTAATTAAC CAAAGAATAG AAGAATTCGC TAGGAACCAA GCCATTTCTA |
| 451 | GATTAGAAGG ACTAAGCAAT CTTTATCAAA TTTACGCAGA ATCTTTTAGA |
| 501 | GAGTGGGAAG CAGATCCTAC TAATCCAGCA TTAAGAGAAG AGATGCGTAT |
| 551 | TCAATTCAAT GACATGAACA GTGCCCTTAC AACCGCTATT CCTCTTTTTG |
| 601 | CAGTTCAAAA TTATCAAGTT CCTCTTTTAT CAGTATATGT TCAAGCTGCA |
| 651 | AATTTACATT TATCAGTTTT GAGAGATGTT TCAGTGTTTG GACAAAGGTG |
| 701 | GGGATTTGAT GCCGCGACTA TCAATAGTCG TTATAATGAT TTAACTAGGC |
| 751 | TTATTGGCAA CTATACAGAT CATGCTGTAC GCTGGTACAA TACGGGATTA |
| 801 | GAGCGTGTAT GGGGACCGGA TTCTAGAGAT TGGATAAGAT ATAATCAATT |
| 851 | TAGAAGAGAA TTAACACTAA CTGTATTAGA TATCGTTTCT CTATTTCCGA |
| 901 | ACTATGATAG TAGAACGTAT CCAATTCGAA CAGTTTCCCA ATTAACAAGA |
| 951 | GAAATTTATA CAAACCCAGT ATTAGAAAAT TTTGATGGTA GTTTTCGAGG |
| 1001 | CTCGGCTCAG GGCATAGAAG GAAGTATTAG GAGTCCACAT TTGATGGATA |
| 1051 | TACTTAACAG TATAACCATC TATACGGATG CTCATAGAGG AGAATATTAT |
| 1101 | TGGTCAGGGC ATCAAATAAT GGCTTCTCCT GTAGGGTTTT CGGGGCCAGA |
| 1151 | ATTCACTTTT CCGCTATATG GAACTATGGG AAATGCAGCT CCACAACAAC |
| 1201 | GTATTGTTGC TCAACTAGGT CAGGGCGTGT ATAGAACATT ATCGTCCACT |
| 1251 | TTATATAGAA GACCTTTTAA TATAGGGATA AATAATCAAC AACTATCTGT |
| 1301 | TCTTGACGGG ACAGAATTTG CTTATGGAAC CTCCTCAAAT TTGCCATCCG |
| 1351 | CTGTATACAG AAAAAGCGGA ACGGTAGATT CGCTGGATGA AATACCGCCA |
| 1401 | CAGAATAACA ACGTGCCACC TAGGCAAGGA TTTAGTCATC CATTAAGCCA |
| 1501 | GAGCTCCTAT GTTCTCTTGG ATACATCGTA GTGCTGAATT TAATAATATA |
| 1551 | ATTCCTTCAT CACAAATTAC ACAAATACCT TTAACAAAAT CTACTAATCT |
| 1601 | TGGCTCTGGA ACTTCTGTCG TTAAAGGACC AGGATTTACA GGAGGAGATA |
| 1651 | TTCTTCGAAG AACTTCACCT GGCCAGATTT CAACCTTAAG AGTAAATATT |
| 1701 | ACTGCACCAT TATCACAAAG ATATCGGGTA AGAATTCGCT ACGCTTCTAC |
| 1751 | CACAAATTTA CAATTCCATA CATCAATTGA CGGAAGACCT ATTAATCAGG |
| 1801 | GGAATTTTTC AGCAACTATG AGTAGTGGGA GTAATTTACA GTCCGGAAGC |
| 1851 | TTTAGGACTG TAGGTTTTAC TACTCCGTTT AACTTTTCAA ATGGATCAAG |
| 1901 | TGTATTTACG TTAAGTGCTC ATGTCTTCAA TTCAGGCAAT GAAGTTTATA |
| 1951 | TAGATCGAAT TGAATTTGTT CCGGCAGAAG TAACCTTTGA GGCAGAATAT |
| 2001 | GATTTAGAAA GAGCACAAAA GGCGGTGAAT GAGCTGTTTA CTTCTTCCAA |
| 2051 | TCAAATCGGG TTAAAAACAG ATGTGACGGA TTATCATATT GATCAAGTAT |
| 2101 | CCAATTTAGT TGAGTGTTTA TCTGATGAAT TTTGTCTGGA TGAAAAAAAA |
| 2151 | GAATTGTCCG AGAAAGTCAA ACATGCGAAG CGACTTAGTG ATGAGCGGAA |
| 2201 | TTTACTTCAA GATCCAAACT TTAGAGGGAT CAATAGACAA CTAGACCGTG |
| 2251 | GCTGGAGAGG AAGTACGGAT ATTACCATCC AAGGAGGCGA TGACGTATTC |
| 2301 | AAAGAGAATT ACGTTACGCT ATTGGGTACC TTTGATGAGT GCTATCCAAC |
| 2351 | GTATTTATAT CAAAAAATAG ATGAGTCGAA ATTAAAAGCC TATACCCGTT |
| 2401 | ACCAATTAAG AGGGTATATC GAAGATAGTC AAGACTTAGA AATCTATTTA |
| 2451 | ATTCGCTACA ATGCCAAACA CGAAACAGTA AATGTGCCAG GTACGGGTTC |
| 2501 | CTTATGGCCG CTTTCAGCCC CAAGTCCAAT CGGAAAATGT GCCCATCATT |
| 2551 | CCCATCATTT CTCCTTGGAC ATTGATGTTG GATGTACAGA CTTAAATGAG |
| 2601 | GACTTAGGTG TATGGGTGAT ATTCAAGATT AAGACGCAAG ATGGCCATGC |
| 2651 | AAGACTAGGA AATCTAGAAT TTCTCGAAGA GAAACCATTA GTAGGAGAAG |
| 2701 | CACTAGCTCG TGTGAAAAGA GCGGAGAAAA AATGGAGAGA CAAACGTGAA |
| 2751 | AAATTGGAAT GGGAAACAAA TATTGTTTAT AAAGAGGCAA AAGAATCTGT |
| 2801 | AGATGCTTTA TTTGTAAACT CTCAATATGA TAGATTACAA GCGGATACCA |
| 2851 | ACATCGCGAT GATTCATGCG GCAGATAAAC GCGTTCATAG CATTCGAGAA |
| 2901 | GCTTATCTGC CTGAGCTGTC TGTGATTCCG GGTGTCAATG CGGCTATTTT |
| 2951 | TGAAGAATTA GAAGGGCGTA TTTTCACTGC ATTCTCCCTA TATGATGCGA |
| 3001 | GAAATGTCAT TAAAAATGGT GATTTTAATA ATGGCTTATC CTGCTGGAAC |
| 3051 | GTGAAAGGGC ATGTAGATGT AGAAGAACAA AACAACCACC GTTCGGTCCT |
| 3101 | TGTTGTTCCG GAATGGGAAG CAGAAGTGTC ACAAGAAGTT CGTGTCTGTC |
| 3151 | CGGGTCGTGG CTATATCCTT CGTGTCACAG CGTACAAGGA GGGATATGGA |
| 3201 | GAAGGTTGCG TAACCATTCA TGAGATCGAG AACAATACAG ACGAACTGAA |
| 3251 | GTTTAGCAAC TGTGTAGAAG AGGAAGTATA TCCAAACAAC ACGGTAACGT |
| 3301 | GTAATGATTA TACTGCGACT CAAGAAGAAT ATGAGGGTAC GTACACTTCT |
| 3351 | CGTAATCGAG GATATGACGG AGCCTATGAA AGCAATTCTT CTGTACCAGC |
| 3401 | TGATTATGCA TCAGCCTATG AAGAAAAAGC ATATACAGAT GGACGAAGAG |
| 3451 | ACAATCCTTG TGAATCTAAC AGAGGATATG GGGATTACAC ACCACTACCA |
| 3501 | GCTGGCTATG TGACAAAAGA ATTAGAGTAC TTCCCAGAAA CCGATAAGGT |
| 3551 | ATGGATTGAG ATCGGAGAAA CGGAAGGAAC ATTCATCGTG GACAGCGTGG |
| 3601 | AATTACTTCT TATGGAGGAA TAATATATGC TTTATAATGT AAGGTGTGCA |
| 3651 | AATAAAGAAT GATTACTGAC TTGTATTGAC AGATAAATAA GGAAATTTTT |
| 3701 | ATATGAATAA AAAACGGGCA TCACTCTTAA AAGAATGATG TCCGTTTTTT |
| 3751 | GTATGATTTA ACGAGTGATA TTTAAATGTT TTTTTTGCGA AGGCTTTACT |
| 3801 | TAACGGGGTA CCGCCACATG CCCATCAACT TAAGAATTTG CACTACCCCC |
| 3851 | AAGTGTCAAA AAACGTTATT CTTTCTAAAA AGCTAGCTAG AAAGGATGAC |
| 3901 | ATTTTTTATG AATCTTTCAA TTCAAGATGA ATTACAACTA TTTTCTGAAG |
| 3951 | AGCTGTATCG TCATTTAACC CCTTCTCTTT TGGAAGAACT CGCTAAAGAA |
| 4001 | TTAGGTTTTG TAAAAAGAAA ACGAAAGTTT TCAGGAAATG AATTAGCTAC |
| 4051 | CATATGTATC TGGGGCAGTC AACGTACAGC GAGTGATTCT CTCGTTCGAC |
| 4101 | TATGCAGTCA ATTACACGCC GCCACAGCAC TCTTATGAGT CCAGAAGGAC |
| 4151 | TCAATAAACG CTTTGATAAA AAAGCGGTTG AATTTTTGAA ATATATTTTT |
| 4201 | TCTGCATTAT GGAAAAGTAA ACTTTGTAAA ACATCAGCCA TTTCAAGTGC |
| 4251 | AGCACTCACG TATTTTCAAC GAATCCGTAT TTTAGATGCG ACGATTTTCC |
| 4301 | AAGTACCGAA ACATTTAGCA CATGTATATC CTGGGTCAGG TGGTTGTGCA |
| 4351 | CAAACTGCAG |

23. A culture of cotton cells according to claim 1.

24. The culture of claim 23 wherein the cotton cells are cells of Gossypium hirsutum, Gossypium arboreum, and Gossypium barbadense.

25. The culture according to claim 24 wherein the plant cells are cells of Gossypium hirsutum.

26. The culture of claim 23 where in the cells are protoplasts.

27. A cotton plant comprising a gene that expresses a polypeptide having substantially the insect toxicity properties of Bacillus thuringiensis crystal protein in sufficient amounts to render the plant toxic to Lepidopteral larvae.

28. The plant of claim 27 wherein the plants are cells of Gossypium hirsutum, Gossypium arboreum, and Gossypium barbadense.

29. The plant according to claim 28 wherein the plant cells are cells of Gossypium hirsutum.

30. A method of producing transformed, embryogenic cotton callus which comprises:
a) contacting a cotton explant with an Agrobacterium vector containing a gene that confers resistance to the antibiotic hygromycin on cotton cells, the period of the contacting being sufficient to transfer the gene to the explant;
b) incubating the transformed explant in a callus growth medium for a period of from about 15 to about 200 hours at a temperature of from about 25 to about 35° C. under a cycle of about 16 hours light and 8 hours dark to develop callus from the explants;
c) contacting the incubated explants with a callus growth medium containing an antibiotic toxic to Agrobacterium for a time sufficient to kill the Agrobacterium;
d) culturing the callus free of Agrobacterium on a callus growth medium;
e) contacting the resulting embryogenic callus with the antibiotic hygromycin in a concentration sufficient to permit selection of callus resistant to the antibiotic hygromycin; and
f) selecting transformed embryogenic callus.

31. The method of claim 30 further comprising the step of germinating the transformed callus and developing plantlets therefrom.

32. The method of claim 30 in which the transformed callus prior to contact with the callus growth medium in step c is rinsed in callus growth medium free of the antibiotic toxic to Agrobacterium.

33. The method of claim 30 wherein the cotton seedling explant is selected from hypocotyl, cotyledon and mixtures thereof.

34. The method of claim 30 wherein the callus growth medium is a Murashige and Skoog medium supplemented with about 1 to about 10 mg/l naphthaleneacetic acid.

35. The method of claim 30 wherein the antibiotic toxic to Agrobacterium is cefotaxime.

36. A method of transforming cotton cells undergoing suspension culture on a callus growth medium which comprises, after a suspension subculture growth cycle; of from about 7 to about 14 days;
a) recovering cells and any embryogenic callus from the callus growth medium;
b) resuspending the cells and embryogenic callus in a callus growth medium containing an Agrobacterium vector having a gene that confers resistance to the antibiotic hygromycin on cotton cells while maintaining suspension growth conditions for a period of time sufficient to transform the suspended cells;
c) recovering the suspended cells from the callus growth medium containing the Agrobacterium;
d) treating the transformed cells and the embryogenic callus with an antibiotic in sufficient concentration to kill the Agrobacterium;
e) contacting the cells and embryogenic callus with the antibiotic hygromycin in order to select the transformed cells and embryogenic callus;
f) filtering the suspension to remove embryogenic callus greater than about 600 microns.

37. The method of claim 36 wherein steps d and e occur before step f.

38. The method of claim 36 wherein steps d and e occur after step f.

39. The method of claim 36 wherein step d occurs before step f and steppe occurs after step f.

40. The method of claim 36 wherein step e occurs before step f and step d occurs after step f.

41. The method of claim 36 wherein the antibiotic of step d is cefotaxime.

42. The method of claim 36 wherein the suspension subculture growth cycle is from about 7 to about 14 days.

43. The method of claim 36 further comprising the step of developing the transformed cotton cells into plantlets.

44. Cotton plants transformed to have resistance to the antibiotic hygromycin.

45. A recombinant DNA vector comprising a plant expressible gene containing the sequence
5′-GTTTT TATTT TTAAT TTTCT TTCAA ATACT TCCA-3′
3′-CAAAA ATAAA AATTA AAAGA AAGTT TATGA AGGT-5′
or a sequence having substantial homology to said sequence, within the 5′ regulatory region for the gene.

46. A plant expressible chimeric gene comprising a
a. a 5′ regulatory region derived from a naturally-occuring plant gene, said gene in nature being regulated by light,
b. a coding sequence encoding a toxin molecule, said toxin being insecticidal to lepidopteran or coleopteran species,
c. a 3′ regulatory region expressible in plants.

47. A chimeric gene of claim 46 wherein the 5′ regulatory region is naturally-occuring in cotton.

48. A chimeric gene of claim 46 wherein the coding sequence is derived from a Bacillus thuringiensis gene.

49. A recombinant DNA vector comprising the gene of claim 46.

50. A recombinant DNA vector comprising the gene of claim 47.

51. A recombinant DNA vector comprising the gene of claim 48.

52. Bacteria carrying a DNA vector of claim 46.

53. Bacteria carrying a DNA vector of claim 47.

54. Bacteria carrying a DNA vector of claim 48.
